(19) United States
(12) Patent Application Publication
(10) Pub. No.: US 2023/0093117 A1
(43) Pub. Date: Mar. 23, 2023

(54) SYSTEMS AND METHODS FOR THE DETECTION AND TREATMENT OF ASPERGILLUS INFECTION

(71) Applicants: Duke University, Durham, NC (US); THE UNITED STATES GOVERNMENT AS REPRESENTED BY THE DEPT OF VETERANS AFFAIRS, Washington, DC (US)

(72) Inventors: Julie STEINBRINK, Durham, NC (US); Micah MCCLAIN, Durham, NC (US); David CORCORAN, Durham, NC (US); Jennifer MODLISZEWSKI, Durham, NC (US); Marisol BETANCOURT-QUIROZ, Durham, NC (US); Aimee ZAAS, Durham, NC (US)

(73) Assignees: Duke University, Durham, NC (US); THE UNITED STATES GOVERNMENT AS REPRESENTED BY THE DEPT OF VETERANS AFFAIRS, Washington, DC (US)

(21) Appl. No.: 17/801,724

(22) PCT Filed: Feb. 23, 2021
(86) PCT No.: PCT/US2021/019281

§ 371 Date: Aug. 23, 2022

Related U.S. Application Data
(60) Provisional application No. 62/980,478, filed on Feb. 24, 2020.

(51) Int. Cl. C12Q 1/6895

(52) U.S. Cl. CPC C12Q 1/6895; C12Q 2600/112; C12Q 2600/158; C12Q 1/6844

(57) ABSTRACT

The present disclosure describes systems, methods, kits, and devices for detecting and treating a fungal infection in a subject. In particular, provided herein are host gene markers that can be used for identifying and treating an Aspergillus infection. The methods, devices, kits, and systems disclosed herein are used to classify subjects based on the expression levels of the identified gene markers. In some embodiments, the Aspergillus infection comprises an infection with Aspergillus fumigatus.

### CROSS-REFERENCES TO RELATED APPLICATIONS

This application claims the benefit of and priority to U.S. Provisional Application No. 62/980,478 filed on Feb. 24, 2020, which is hereby incorporated by reference in its entirety.

### BACKGROUND

Invasive aspergillosis (IA) continues to be a significant cause of morbidity and mortality in immunocompromised patients. However, diagnosis of invasive fungal infection remains difficult. This is evidenced by the that in clinical practice and research, invasive fungal diagnoses are categorized as ‘possible,’ ‘probable,’ and ‘proven’ based on a combination of host factors, clinical evidence, and microbiologic evaluation.

The gold standard for diagnosis of ‘proven’ invasive fungal infection is identification of fungal tissue invasion on histopathologic examination or culture from a typically sterile site. However, obtaining tissue for diagnosis is often complicated due to comorbid patient risk factors, and thus ‘proven’ infection is challenging to confirm. For IA in particular, blood cultures are rarely helpful even in severe disease, and cultures of sputum or BAL fluid suffer simultaneously from low sensitivity and low specificity.

Serum and bronchoalveolar lavage (BAL) galactomannan can function as indirect indicators of infection, and are relatively specific for Aspergillus though with variable sensitivity. In meta-analyses of immunocompromised patients, serum galactomannan sensitivity ranged from 22% to 82% depending on the patient population under analysis. A number of other factors can also affect the accuracy of testing, including prior antifungal therapy, certain blood products, and intravenous immunoglobulin.

Serum beta-d-glucan (BDG) is another non-invasive serologic marker for fungal infection that detects the presence of 1,3-beta-d-glucan, a component of many fungal cell walls. However, it is not specific for Aspergillus, and can also be affected by multiple factors including intravenous immunoglobulin, albumin, and hemodialysis, leading to false-positive results. In a meta-analysis of immunocompromised population, sensitivity was 80% with a specificity of only 63%.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim.

Provided herein is a method of treating an Aspergillus infection in a subject, the method comprising: (a) measuring, the gene expression levels of two or more aspergillosis versus reference (“AvR”) genes selected from the group consisting of gene markers listed in Table 2 in a biological sample obtained from the subject, and (b) administering an effective amount of an antifungal treatment to the subject identified as having an Aspergillus infection based on comparison of the gene expression levels of the two or more AvR genes with reference gene expression levels of the AvR genes in a reference sample having a known Aspergillus infection classification.

Also provided is a method of treating an Aspergillus infection in a subject, the method comprising: (a) selecting a subject who has been classified as having an Aspergillus infection based on the gene expression levels of two or more aspergillosis versus reference (“AvR”) genes selected from the group consisting of gene markers listed in Table 2 relative to reference expression levels determined for the AvR genes in a reference sample having a known Aspergillus infection classification, and (b) administering to the subject an effective amount of an antifungal treatment.

Also provided is a method of determining the presence of an Aspergillus infection in a subject, the method comprising: (a) measuring, the gene expression levels of two or more aspergillosis versus reference (“AvR”) genes selected from the group consisting of gene markers listed in Table 2 in a biological sample obtained from the subject; and (b) identifying the subject as having an Aspergillus infection based on comparison of the gene expression levels of the two or more AvR genes in the biological sample to reference expression levels determined for the AvR genes in a reference sample having a known Aspergillus infection classification.

Also provided are systems, devices, kits and panels useful for the treatment and diagnosis of an Aspergillus infection in a subject. In some aspects, the systems, devices, kits and panels provided herein can be used to measure gene expression levels of two or more aspergillosis versus reference (“AvR”) genes selected from the group consisting of gene markers listed in Table 2.

In some instances, the provided methods, systems, devices, kits and panel are used to detect and treat an Aspergillus infection in immunocompromised subjects. In some instances, the Aspergillus infection is caused by Aspergillus fumigatus.

### BRIEF DESCRIPTION OF THE DRAWINGS

FIG. 1 shows a schematic illustration of the experimental design according to certain aspects of this disclosure. Breakdown of animals is by inhalational Aspergillus exposure (present or absent) and type of immunosuppression (none, cyclophosphamide, and corticosteroids). A total of 60 male BALB/c mice were separated into two experimental groups. Mice were divided into groups based on Aspergillus fumigatus exposure: ‘No Aspergillus’ exposure group, i.e., placebo group (n=30) and ‘Inhalational Aspergillus’ exposure group (n=30). Within each Aspergillus exposure group mice were divided into groups based on immunosuppressed state. The Aspergillus exposure group consisted of healthy mice, i.e., no immunosuppression (no immunosuppressive drug) (n=11), mice exposed to corticosteroids (n=11), and mice exposed to cyclophosphamide (n=8). The placebo group consisted of healthy mice (n=10), mice exposed to corticosteroids (n=10), and mice exposed to cyclophosphamide (n=10).

FIG. 2A shows a bar graph illustrating mean mouse weights in grams at day +4 after inhalational Aspergillus exposure based on experimental group (no Aspergillus/no immunosuppression, no Aspergillus/corticosteroids, no Aspergillus/cyclophosphamide, Aspergillus/no immunosuppression, Aspergillus/corticosteroids, Aspergillus/cyclophosphamide) according to certain aspects of this disclosure. Error bars represent standard deviation. AF=Aspergillus fumigatus, IS=immunosuppression, Cyclo=cyclophosphamide, CA=corticosteroids.

FIG. 2B shows a bar graph illustrating mean mouse weights in grams at day +4 comparing those with inhalational Aspergillus exposure and those without according to certain aspects of this disclosure.

FIG. 2C shows a bar graph illustrating mean mouse weights in grams at day +4 comparing those mice who received immunosuppression to those who did not according to certain aspects of this disclosure.

FIG. 2D shows a bar graph illustrating mean mouse lung fungal burden, measured in colony-forming units/gram (CFU/g) at time of day +4 after inhalational Aspergillus infection comparing those with no suppression, cyclophosphamide immunosuppression, and corticosteroid immunosuppression according to certain aspects of this disclosure.

FIG. 3A shows full model predictions based on control (no immunosuppressive drug) data according to certain aspects of this disclosure. This model is referred to in the Examples as the Control model/analysis and generated the gene markers of Classifier 1, which are listed in Table 1. The graph shows the predicted infection status for each condition (No drug, Cyclophosphamide, and Steroids). All samples from the no immunosuppression group were used in model training. The predictions represent a best-case scenario as the entire control data set (i.e., no immunosuppressive drug) was used to train the model.

FIG. 3B shows leave-one-out cross-validation results of the control data set described in FIG. 3A showing model robustness and expected outcome if the model was run on a validation cohort according to certain aspects of this disclosure.

FIG. 4A shows full model predictions for each drug therapy condition (No drug, Cyclophosphamide, and Steroids) according to certain aspects of this disclosure. This model is referred to in the Examples as the Complete model/analysis and generated the gene markers of Classifier 2, which are listed in Table 2. All samples were used in model training. All predictions represent a best-case scenario as the same data was used in training and testing the model.

FIG. 4B shows ten-fold cross-validation results showing model robustness and expected outcome if the model was run on a validation cohort according to certain aspects of this disclosure.

FIG. 4D shows ROC curves of cross-validation performance of the model in the setting of corticosteroid exposure, with an AUC of 0.9 according to certain aspects of this disclosure.

FIG. 4E shows ROC curves of cross-validation performance of the model in the setting of cyclophosphamide exposure, with an AUC of 1 according to certain aspects of this disclosure.

FIG. 4F shows ROC curves of cross-validation performance of the model in the setting of no immunosuppression, with an AUC of 0.92 according to certain aspects of this disclosure.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs. All publications mentioned herein are incorporated herein by reference for the purpose of describing and disclosing devices, compositions, formulations, and methodologies which are described in the publication and which might be used in connection with the presently described invention.

### 1. Introduction

Aspergillosis is an infection, usually of the lungs, caused by the fungus Aspergillus. Among the human pathogenic species of Aspergillus, A. fumigatus is the primary causative agent of human infections. In individuals with altered lung function such as asthma and cystic fibrosis patients, aspergilli can cause allergic bronchopulmonary aspergillosis, a hypersensitive response to fungal components. Noninvasive aspergillomas (mass-like fungus balls) may form following repeated exposure to conidia and target preexisting lung cavities such as the healed lesions in tuberculosis patients. Invasive aspergillosis (IA) is the most serious of Aspergillus-related diseases. IA occurs when the infection spreads rapidly from the lungs to the brain, heart, kidneys or skin. Poorly controlled IA can cause widespread organ damage such as kidney failure or liver failure, and may reach mortality rates as high as 90% in some patient groups. Those most at risk for this life-threatening disease are immunocompromised individuals with hematological malignancies such as leukemia; solid-organ and hematopoietic stem cell transplant patients; patients on prolonged corticosteroid therapy, which is commonly utilized for the prevention and/or treatment of graft-versus-host disease in transplant patients; individuals with genetic immunodeficiencies such as chronic granulomatous disease (CGD); and individuals infected with human immunodeficiency virus.

Among the factors that are responsible for the increased risk of IA, neutropenia and corticosteroid-induced immunosuppression are the most important ones. Prolonged neutropenia is often the result of highly cytotoxic therapies such as cyclophosphamide, which is used for transplant patients or those with hematological diseases. Cyclophosphamide interferes with cellular replication, depleting circulating white blood cells including neutrophils. The lack of inflammatory infiltrates results in low levels of inflammation, affecting the body's major defense against acute infection (e.g., fungal infection). Another high risk group includes individuals on corticosteroid therapy such as allogeneic transplant patients receiving corticosteroids for prophylaxis or treatment of graft-versus-host disease. Corticosteroids have significant consequences for phagocyte function, including but not limited to the impairment of phagocytosis, phagocyte oxidative burst, production of cytokines and chemokines, and cellular migration. For example, it has been shown that corticosteroids impair the functional ability of phagocytes to kill fungi.

As discussed herein, current fungal diagnostics remain limited. For example, existing methods suffer from challenges with test sensitivity and susceptibility to false positive results. Thus, an improved approach to IA testing and screening is needed. Specifically, methods that allow sensitive and accurate identification of infection in patients, particularly immunocompromised patients, would improve clinical outcome by administering timely and effective treatment regimen.

The present disclosure addresses the need for an improved Aspergillus infection diagnosis and treatment by providing a host gene expression signature reflective of an Aspergillus infection. Analysis of host gene expression levels has emerged as a sensitive approach for investigating the host's response to disease. For example, transcriptome analysis of host responses to infection can be used to reveal systemic changes in host gene expression profiles caused by the infection. By comparing such transcriptomic profiles in samples from subjects with the infection versus those without, it is possible to identify genes that differ in their expression between the groups, and thus are part of the disease signature. The transcriptional signatures can be used as diagnostic tools allowing the classification of individuals based on the expression profile of the identified gene markers.

Described herein is the discovery that the expression level of certain host genes can predict whether a subject has an Aspergillus infection. In particular, the disclosure provides a host gene signature that can be used as a diagnostic tool across immunocompromised states. The identified gene signature can be used to identify an Aspergillus infection in a subject so that appropriate treatment can be administered promptly. Accordingly, this disclosure provides methods of treating a subject (e.g., an immunocompromised patient) by determining the presence of an Aspergillus infection based on the expression levels of the identified host gene markers, and administering to the subject an antifungal treatment if the subject has been determined to have an Aspergillus infection. Also provided is a method of diagnosing an Aspergillus infection in immunocompromised individuals. For example, this disclosure provides methods of determining the presence of an Aspergillus infection in a subject (such as an immunocompromised patient) by measuring and analyzing the expression level of the identified gene markers.

The systems, methods, and devices described herein use a plurality of host gene markers comprising gene markers selected form the group of genes listed in Table 1 or Table 2 as described below. The plurality of gene markers may be referred to as a gene marker panel. In some embodiments, the expression levels of two or more of these genes may be altered (e.g., increase or decrease) in a subject as a result of an Aspergillus infection. In some embodiments the gene markers provided herein may be differentially expressed in a subject having an Aspergillus infection. As used herein, the term “differentially expressed” refers to differences in the expression level or abundance (i.e., in the quantity and/or the frequency) of a gene product (e.g., RNA) present in a sample taken from a subject having an Aspergillus infection as compared to a reference sample obtained from a subject not infected with Aspergillus. For example, the mRNA transcript levels of a gene marker may be present at an elevated level or at a decreased level in samples from subjects with an Aspergillus infection compared to reference samples from subjects that are not infected with Aspergillus. In some embodiments, differential expression of a plurality of the gene markers in a biological sample from a subject relative to in a reference sample obtained from a subject not infected with Aspergillus is indicative that the subject has an Aspergillus infection.

As used herein, the term “fungal infection” refers to any disease caused by a fungus in a subject. In some embodiments, the fungal infection causes respiratory illnesses. In some embodiments, the fungal infection is caused by a fungus in the genus Aspergillus. As used herein, the term “Aspergillus” refers to the genus fungus whose spores are present in the air we breathe, but does not normally cause illness. In some embodiments, the Aspergillus comprises Aspergillus fumigatus. In those people with a weakened immune system, damaged lungs or with allergies, Aspergillus can cause disease. As used herein, there term “Aspergillus infection” refers to those disease states caused by Aspergillus. Examples of Aspergillus infections include, but are not limited to, invasive aspergillosis (IA), allergic bronchopulmonary aspergillosis (ABPA), chronic pulmonary aspergillosis (CPA), aspergilloma, and the like.

As used herein, the term “signature” as used herein and refer to a set of biological analytes and the measurable quantities of said analytes whose expression level signifies the presence or absence of the specified biological state (e.g., a fungal infection). These signatures are discovered in a plurality of subjects with known infection status (e.g. a confirmed infection with a fungus (e.g., Aspergillus) or lacking a fungal infection), and are discriminative (individually or jointly) of one or more categories or outcomes of interest. These measurable quantities, also known as biological markers or host gene markers, can be (but not limited to) gene expression levels, protein or peptide levels, or metabolite levels.

In some embodiments, a “signature” may comprise a particular combination of gene products whose expression levels, when incorporated into a classifier as taught herein, discriminate a condition such as a fungal infection. The term “fungal gene product expression levels” and “fungal signature” are used interchangeably and refer to the level of gene products, for example, such as those proteins and/or peptides as described herein. The altered expression of one or more of these gene products is indicative of the subject having a fungal infection. In some embodiments, the signature is able to distinguish individuals with infection due to a fungus from individuals lacking a fungal infection. In other embodiments, the signature is able to distinguish individuals infected with Aspergillus or infected with a different fungal infection.

The term “gene” means the segment of DNA involved in producing a polypeptide chain or transcribed RNA product. It may include regions preceding and following the coding region (leader and trailer) as well as intervening sequences (introns) between individual coding segments (exons).

The terms “gene marker” or “host gene marker”, as used herein, refer to a gene and its expression level found in a subject that can be used for diagnosis and/or other classification purposes. Such markers may also be referred to as “host biomarkers.” Such gene markers can be identified by embodiments of the present disclosure. The host gene marker refers to a gene or a portion thereof that has differential expression in a subject infected with Aspergillus (e.g., with Aspergillus fumigatus) as compared to a subject who is not infected with Aspergillus.

As used herein, the term “gene product” refers to any biochemical material resulting from the expression of a gene. Examples include, but are not limited to, nucleic acids such as RNA and mRNA, proteins, component peptides, expressed proteomes, epitopes, and any subsets thereof, and combinations thereof. In certain embodiments, the gene product comprises proteins and/or component peptides (e.g., all expressed proteins and/or peptides, or expressed proteome, epitopes or a subset thereof). In some embodiments, the gene product is RNA, particularly mRNA.

The term “genetic material” refers to a material used to store genetic information in the nuclei or mitochondria of an organism's cells. Examples of genetic material include, but are not limited to double-stranded and single-stranded DNA, cDNA, RNA, mRNA, or their encoded products.

The term “nucleic acid” or “polynucleotide” refers to a deoxyribonucleic acid (DNA) or ribonucleic acid (RNA) and a polymer thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), copy number variants, and complementary sequences as well as the sequence explicitly indicated.

The terms “peptide,” “polypeptide,” and “protein” are used interchangeably herein to refer to a polymer of amino acid residues.

As used herein, the term “indicative” when used with a gene product expression levels, means that the expression levels are up-regulated or down-regulated, altered, or changed compared to the expression levels in alternative biological states or in reference samples (e.g., uninfected). The term “indicative” when used with protein and/or peptide levels means that the expression levels are higher or lower, increased or decreased, altered, or changed compared to the standard protein levels.

### 2. Gene Signatures

As described in the Examples, the inventors determined changes in the transcriptome of subjects infected with Aspergillus and identified host genes whose expression levels were altered by an Aspergillus infection as compared to uninfected subjects. These identified gene markers are hereafter referred to as aspergillosis versus reference (“AvR”) genes. In particular, two sets of AvR genes were identified that showed differential expression in subjects having an Aspergillus infection as compared to uninfected subjects. One set of AvR genes (referenced herein as “classifier 1”) includes gene markers that exhibit altered expression level in the absence of immunosuppression. Another set of AvR genes (referenced herein as “classifier 2”) includes gene markers that show altered expression levels across subjects with different immunosuppressive states. The identified gene markers perform well in distinguishing infected subjects from non-infected subjects. The identified genes markers may be used in gene panels for a given diagnostic and/or treatment method. This section summarizes the identified gene markers and provides examples of gene marker panels.

The gene markers of classifier 1 were identified through the analyses described in Example 3 and are listed in Table 1. Thus, provided herein are the gene markers listed in Table 1 and their diagnostic and therapeutic uses for assessing and treating an Aspergillus infection. In some approaches, the expression level of the gene markers of classifier 1 may be used for determining the presence of an Aspergillus infection in immunocompetent individuals. In certain embodiments, the Aspergillus infection is an Aspergillus fumigatus infection, i.e., an infection caused by Aspergillus fumigatus.

The genes of classifier 2 were identified through the analyses described in Section Example 5 and are listed in Table 2. Accordingly, provided herein are the gene markers shown in Table 2 and their diagnostic and therapeutic uses for assessing and treating an Aspergillus infection. In some approaches, the expression level of the gene markers of classifier 2 may be used for determining the presence of an Aspergillus infection in immunocompromised individuals. In certain embodiments, the Aspergillus infection is an Aspergillus fumigatus infection, i.e., an infection caused by Aspergillus fumigatus.

As described in the Examples the two classifiers distinguished Aspergillus infected mice from non-infected mice. Classification accuracy was determined using an area under the curve (AUC) measure. The “area under curve” or “AUC” refers to area under a ROC curve. AUC under a ROC curve is a measure of accuracy. An area of 1 represents a perfect test, whereas an area of 0.5 represents an insignificant test. A preferred AUC may be between 0.700 and 1. For example, a preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

In some embodiments, the host gene markers provided in Tables 1 and 2 have a differential expression in a subject that has an Aspergillus infection as compared to a subject that does not have an Aspergillus infection. In some aspects of this disclosure, a plurality of the gene markers listed in Table 1 or Table 2 can be used to identify and diagnose an Aspergillus infection in a subject. The gene markers that exhibit increased expression levels in infected subjects as compared to non-infected subjects are listed as upregulated genes (referred to as “upregulated genes” herein). The gene markers that exhibit decreased expression levels compared to non-infected subjects are listed as downregulated genes (referred to as “downregulated genes” herein). Sequence identifiers are provided, but it will be understood that gene markers include variants (e.g., polymorphic variants, etc.) of the identified genes.

In some embodiments, the expression level of two or more AvR genes selected from the group consisting of AvR genes listed in Table 1 is measured. In some embodiments, the expression level of two or more AvR genes selected from the group consisting of AvR genes listed in Table 2 is measured. In some embodiments, the expression level of a plurality of gene markers selected from the group consisting of gene markers listed in Table 1 is measured. In some embodiments, the expression level of 2, 5, 10, 15, 20, 25, 30, 35, 40, 45, 50, 55, 60, 65, 70, 75, 80, 85, 90, 95, 100, 105, 110, 115, 120, 125, 130, 135, 140, 145 or 146 gene markers selected from the group consisting of gene markers listed in Table 1 is measured. In some embodiments, the expression level of 10 gene markers selected from the group consisting of gene markers listed in Table 1 is measured. In some embodiments, the expression level of 20 gene markers selected from the group consisting of gene markers listed in Table 1 is measured. In some embodiments, the expression level of 50 gene markers selected from the group consisting of gene markers listed in Table 1 is measured. In some embodiments, the expression level of 100 gene markers selected from the group consisting of gene markers listed in Table 1 is measured. In some embodiments, the expression level of 110 gene markers selected from the group consisting of gene markers listed in Table 1 is measured. In some embodiments, the expression level of 120 gene markers selected from the group consisting of gene markers listed in Table 1 is measured. In some embodiments, the expression level of 130 gene markers selected from the group consisting of gene markers listed in Table 1 is measured. In some embodiments, the expression level of 140 gene markers selected from the group consisting of gene markers listed in Table 1 is measured. In some embodiments, the expression level of 146 gene markers selected from the group consisting of gene markers listed in Table 1 is measured.

In some embodiments, the expression level of a plurality of gene markers selected from the group consisting of gene markers listed in Table 2 is measured. In some embodiments, the expression level of 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 187 gene markers selected from the group consisting of gene markers listed in Table 2 is measured. In some embodiments, the expression level of 10 gene markers selected from the group consisting of gene markers listed in Table 2 is measured. In some embodiments, the expression level of 20 gene markers selected from the group consisting of gene markers listed in Table 2 is measured. In some embodiments, the expression level of 50 gene markers selected from the group consisting of gene markers listed in Table 2 is measured. In some embodiments, the expression level of 100 gene markers selected from the group consisting of gene markers listed in Table 2 is measured. In some embodiments, the expression level of 110 gene markers selected from the group consisting of gene markers listed in Table 2 is measured. In some embodiments, the expression level of 120 gene markers selected from the group consisting of gene markers listed in Table 2 is measured. In some embodiments, the expression level of 130 gene markers selected from the group consisting of gene markers listed in Table 2 is measured. In some embodiments, the expression level of 140 gene markers selected from the group consisting of gene markers listed in Table 2 is measured. In some embodiments, the expression level of 146 gene markers selected from the group consisting of gene markers listed in Table 2 is measured. In some embodiments, the expression level of 150 gene markers selected from the group consisting of gene markers listed in Table 2 is measured. In some embodiments, the expression level of 160 gene markers selected from the group consisting of gene markers listed in Table 2 is measured. In some embodiments, the expression level of 170 gene markers selected from the group consisting of gene markers listed in Table 2 is measured. In some embodiments, the expression level of 180 gene markers selected from the group consisting of gene markers listed in Table 2 is measured. In some embodiments, the expression level of 187 gene markers selected from the group consisting of gene markers listed in Table 2 is measured.

The plurality of gene markers may be referred to as a gene marker panel and may comprise any suitable number of gene markers selected from the gene markers listed in Table 1 or Table 2. In some instances, a gene marker panel may comprise between 2 to 146 gene markers, inclusive, including for example 2, 5, 10, 15, 20, 25, 30, 35, 40, 45, 50, 55, 60, 65, 70, 75, 80, 85, 90, 95, 100, 105, 110, 115, 120, 125, 130, 135, 140, 145 or 146 gene markers selected from the gene markers listed in Table 1. In some instances, the gene marker panel may comprise at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140 or more gene markers selected from the gene markers as listed in Table 1. In some instances, the gene marker panel may comprise all genes listed in Table 1. In some instances, the gene marker panel comprises 146 genes consisting of the group of genes listed in Table 2. In some instances, a gene marker panel may comprise between 2 to 187 gene markers, inclusive, including for example 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 187 gene markers selected from the gene markers listed in Table 2. In some instances, the gene marker panel may comprise at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180 or more gene markers selected from the gene markers as listed in Table 2. In some instances, the gene marker panel may comprise all genes listed in Table 2. In some instances, the gene marker panel comprises 187 genes consisting of the group of genes listed in Table 2.

In some aspects, increased expression levels of upregulated genes identified in Table 1 and decreased expression levels of downregulated genes identified in Table 1 as compared to reference gene expression levels determined from a plurality of reference samples not infected with Aspergillus indicate that the subject has an Aspergillus infection. In some aspects, increased expression levels of upregulated genes identified in Table 2 and decreased expression levels of downregulated genes identified in Table 2 as compared to reference gene expression levels determined from a plurality of reference samples not infected with Aspergillus indicate that the subject has an Aspergillus infection. In some embodiments, the gene markers are selected from one or more gene markers up-regulated, down-regulated, or over-expressed by 5-fold, 4.5-fold, 4-fold, 3.9-fold, 3.8-fold, 3.7-fold, 3.6-fold, 3.5 fold, 3-fold, 2.9 fold, 2.8 fold, 2.7 fold, 2.6 fold, 2.5 fold, 2.4 fold, 2.3 fold, 2.2 fold, 2.1 fold, 2-fold, 1.9 fold, 1.8 fold, 1.7 fold, 1.6 fold, 1.5 fold, 1.4 fold, 1.3 fold, 1.2 fold, 1.1 fold, 1-fold, 0.9 fold, 0.8 fold, 0.7 fold, 0.6 fold, or 0.5-fold in a subject having an Aspergillus infection, when compared to a reference sample obtained from a subject not infected with Aspergillus. In some embodiments, if gene marker as identified in Table 1 or 2 display a differential expression level in the biological sample from the subject relative to reference gene expression levels in the reference sample obtained from a subject not infected with Aspergillus, i.e., higher or lower than the reference gene expression level in the reference sample, then the subject may have an Aspergillus infection. In some embodiments, the differential expression is up to 3-fold, up to fold 2-fold, at least 1-fold, or at least 0.5-fold. In some embodiments, the differential expression is 0.5 fold to 3 fold.

### 3. Methods of Measuring Gene Expression Levels

Techniques and methods for measuring the expression levels of genes are available in the art. Thus, measuring the expression level of genes listed in Tables 1 or Table 2 may be accomplished by using any suitable platform or technology. For example, gene expression levels may be measured using platforms that are based on polymerase chain reaction (PCR) methods (including reverse transcription-PCR (RT-PCR)), peptide or nucleic acid sequencing methods, hybridization capture methods, microarray analysis, mass spectrometry (MS), Northern blot, serial analysis of gene expression (SAGE), or immunoassays. These methods are described, for example, in Sambrook and Russel (2001), Molecular Cloning: A Laboratory Manual, 3rd Edition, Cold Spring Harbor, N.Y.: Cold Spring Harbor Laboratory Press; Velculescu et al., 1995, Science 270:484-7.

“Platform” or “technology” as used herein refers to an apparatus (e.g., instrument and associated parts, computer, computer-readable media comprising one or more databases as taught herein, reagents, etc.) that may be used to measure a signature, e.g., gene expression levels, in accordance with the present disclosure. Examples of platforms include, but are not limited to, an array platform, a nucleic acid sequencing platform, a thermocycler platform (e.g., multiplexed and/or real-time PCR platform, e.g., a TagMan® Low Density Array (TLDA), a Biocartis Idylla™ sample-to-result technology, etc.), a gene product hybridization or capture platform (e.g., a nucleic acid, protein, and/or peptide hybridization or capture platform), a multi-signal coded (e.g., fluorescence) detector platform, etc., a mass spectrometry platform, an amino acid sequencing platform, a magnetic resonance platform (e.g., the T2 Biosystem® T2 Magnetic Resonance (T2MR®) technology; electrospray ionization (ESI), matrix-assisted laser desorbtion/ionization (MALDI), etc.), and combinations thereof. In some embodiments, the platforms may comprise a protein and/or peptide hybridization or capture platform, a multi-signal coded (e.g., fluorescence) detector platform, etc., an amino acid sequencing platform and combinations thereof.

In some embodiments, the platform is configured to measure gene product expression levels semi-quantitatively, that is, rather than measuring in discrete or absolute expression, the expression levels are measured as an estimate and/or relative to each other or a specified marker or markers (e.g., expression of another, “standard” or “reference,” gene or gene product).

The terms “array,” “microarray” and “micro array” are interchangeable and refer to an arrangement of a collection of reagents presented on a substrate. Any type of array can be utilized in the methods provided herein. For example, arrays can be on a solid “planar” substrate (a solid phase array), such as a glass slide, or on a semi-solid substrate, such as nitrocellulose membrane. Arrays can also be presented on beads, i.e., a bead array. These beads are typically microscopic and may be made of, e.g., polystyrene. The array can also be presented on nanoparticles, which may be made of, e.g., particularly gold, but also silver, palladium, or platinum. Magnetic nanoparticles may also be used. Other examples include nuclear magnetic resonance microcoils. The analyte specific reagents can be antibody or antibody fragments or nucleic acid aptamers or probes, for example. The arrays may additionally comprise other compounds, such as nucleic acids, peptides, proteins, cells, chemicals, carbohydrates, and the like that specifically bind nucleic acids, proteins, peptides, or metabolites.

An array platform may include, for example, the MesoScaleDiscovery (MSD) platform for measurement of multiple analytes per well, configured as antibody “spots” in each assay well. The MSD platform utilizes chemiluminescent reagents activated upon electrical stimulation, or “electrochemiluminescence” detection.

A hybridization and multi-signal coded detector platform includes, for example, NanoString nCounter® technology, in which hybridization of a color-coded barcode attached to a target-specific probe (e.g., barcoded antibody probe) is detected; and Luminex® technology, in which microsphere beads are color coded and coated with a target-specific reagents (e.g., color-coded beads coated with analyte-specific antibody) probe for detection.

In some approaches, polymerase chain reaction (PCR) may be used to measure the gene expression levels of the gene markers provided herein. PCR-based methods that may be used include but are not limited to quantitative PCR (qPCR or real-time PCR), reverse transcriptase PCR (RT-PCR), and digital PCR. PCR methods are well known in the art, and are described, for example, in Innis et al., eds., PCR Protocols: A Guide To Methods And Applications, Academic Press Inc., San Diego, Calif. (1990); see Sambrook and Russel (2001), Molecular Cloning: A Laboratory Manual, 3rd Edition, Cold Spring Harbor, N.Y.: Cold Spring Harbor Laboratory Press; Chapter 8: In vitro Amplification of DNA by the Polymerase Chain Reaction; PCR Technology: Principles and Applications for DNA Amplification (ed. H. A. Erlich, Freeman Press, N.Y., N.Y., 1992, herein incorporated by reference in their entirety. See also, e.g. Real-Time PCR: Current Technology and Applications, Logan, Edwards, and Saunders eds., Caister Academic Press, 2009; Joyce (2002), “Quantitative RT-PCR. A review of current methodologies,” Methods Mol. Biol. 193. pp. 83-92; Bustin et al. (2005), “Quantitative real-time RT-PCR—a perspective,” J. Mol. Endocrinol. 34 (3): 597-601; Bustin (2000), “Absolute quantification of mRNA using real-time reverse transcription polymerase chain reaction assays,” J. Mol. Endocrinol. 25 (2): 169-93; Deepak et al. (2007), “Real-Time PCR: Revolutionizing Detection and Expression Analysis of Genes”. Curr. Genomics. 8 (4): 234-51; Gause et al. (1994). “The use of the PCR to quantitate gene expression”. PCR Methods Appl. 3 (6): S123-35.

Accordingly, in some approaches measuring the expression level of the two or more genes shown in Table 1 and 2 comprises performing PCR (e.g., qRT-PCR). In some approaches, RNA probes may be developed for the gene markers listed in Table 1 and/or Table 2. The RNA may be measured by PCR (e.g., RT-PCR). The RNA expression may be measured and compared to reference expression levels for these selected genes. The PCR may be performed by using at least one set of oligonucleotide primers comprising a forward primer and a reverse primer capable of amplifying a polynucleotide sequence of the gene. Methods for the design and/or production of nucleotide primers are generally known in the art, and are described in e.g., Sambrook et al. (2001) Molecular Cloning: A Laboratory Manual (3rd ed., Cold Spring Harbor Laboratory Press, Plainview, N.Y.); Ausubel F. M. et al. (Eds) Current Protocols in Molecular Biology (2007), John Wiley and Sons, Inc; Molecular Cloning: A Laboratory Manual, 4th ed., Green and Sambrook, 2012). Nucleotide primers and probes may be prepared, for example, by chemical synthesis techniques for example, the phosphodiester and phosphotriester methods (see for example Narang S. A. et al. (1979) Meth. Enzymol. 68:90; Brown, E. L. (1979) et al. Meth. Enzymol. 68:109; and U.S. Pat. No. 4,356,270), the diethylphosphoramidite method (see Beaucage S. L et al. (1981) Tetrahedron Letters, 22:1859-1862). Oligonucleotide primers are typically being between 5-80 nucleotides in length, e.g., between 10-50 nucleotides in length, or between 15-30 nucleotides in length. Any appropriate length of sequence may be used such as 10, 11, 12, 13, 14, 15, 16, 17, 18, 19, 20, 21, 22, 23, 24, 25, 26, 27, 28, 29, or 30 nucleotides or more.

In some approaches, the gene expression levels may be measured using nucleic acid sequencing technologies, such as next generation sequencing platforms (e.g., RNA-Seq). RNA-SEQ uses next-generation sequencing (NGS) for the detection and quantification of RNA in a biological sample at a given moment in time. An RNA library is prepared, transcribed, fragmented, sequenced, reassembled and the sequence or sequences of interest quantified. NGS methods are well known in the art and described e.g., in Mortazavi et al., Nat. Methods 5: 621-628, 2008; Karl et al. (2009), “Next-Generation Sequencing: From Basic Research to Diagnostics,” Clinical Chemistry. 55 (4): 641-658; Wang et al. (2009), “RNA-Seq: a revolutionary tool for transcriptomics,” Nature Reviews. Genetics. 10 (1): 57-63; Kukurba and Montgomery (2015), “RNA Sequencing and Analysis”, Cold Spring Harbor Protocols., (11): 951-69. In some approaches, whole transcriptome shotgun sequencing may be used to measure gene expression levels. In some approaches, metagenomics NGS (mNGS) may be used to measure gene expression levels. See e.g., Chiu and Miller (2019), “Clinical metagenomics,” Nature Reviews Genetics, 20 (6): 341-355; Maljkovic et al. (2019), “Next Generation Sequencing and Bioinformatics Methodologies for Infectious Disease Research and Public Health: Approaches, Applications, and Considerations for Development of Laboratory Capacity,” The Journal of Infectious Diseases: jiz286; Wilson et al. (2019), “Clinical metagenomic sequencing for diagnosis of meningitis and encephalitis,” N. Engl. J. Med. 380, 2327-2340. Exemplary sequencing platforms suitable for use according to the methods include, e.g., ILLUMINA® sequencing (e.g., HiSeq, MiSeq), SOLID® sequencing, ION TORRENT® sequencing, and SMRT® sequencing and those commercialized by Roche 454 Life Sciences (GS systems).

In some embodiments, semi-quantitative measuring includes immunodetection methods including ELISA or protein arrays, which utilize analyte specific immuno-reagents to provide specificity for particular protein or peptide sequence and/or structure, coupled with signal detection modalities such as fluorescence or luminescence to provide the estimated or relative expression levels of the genes within the signature.

Gene products may also be measured using mass spectrometry. For example, protein and/or peptide mass spectrometry (MS) utilizes instruments capable of accurate mass determination and includes a variety of instruments and methods. MS provides a tool for comprehensive proteomic survey of biological samples, as well as for targeted identification and measurement of specific protein, peptides, or metabolites. Many technical variations exist that differ in specificity, sensitivity, dynamic range, throughput, and cost, though each involve the conversion of proteins into component peptide fragments followed by their volatilization and measurement of their mass-to-charge ratio and intensity, paired with comparison to protein databases for identification. MS methods are often paired with pre-fractionation or purification (e.g. liquid chromatography) to reduce complexity of samples. One variation of targeted MS measurement, multiple/selective reaction monitoring (MRM/SRM), provides significant improvements in sensitivity and coefficients of variation, and provides opportunity for targeted measurement of multiple protein or peptide analytes. In some embodiments, the measurement by MS is performed using two primary methods: electrospray ionization (ESI) and matrix-assisted laser desorbtion/ionization (MALDI). Proteins may be analyzed either as “top-down” approach characterizing intact proteins, or a “bottom up” approach characterizing digested protein fragments or peptides. Protein or peptide MS may be performed in conjunction with up-front methods to reduce complexity of biological samples, such as gel electrophoresis or liquid chromatography. Resulting MS data can be used to identify and quantify specific proteins and/or peptides. MS is also widely accepted as one of the most accurate methods to detect nucleic acids. Using matrix-assisted laser desorption/ionization time-of-flight (MALDI-TOF) MS, a mass resolution of 1 per 1000 and the detection of low femtomole quantities of DNA can be achieved routinely. MS can also be used to analyze mixtures of different nucleic acid fragments without the use of any label because of the mass differences of the nucleobases. The separation of the fragments before MS measurements is also typically not required. The minimum sample volume requirement (a few nanoliters) and fast sample processing time (less than 10 sec) has led to the the availability of automatic high-throughput MS systems that include sample preparation.

Gene products may also be measured using an immunoassay, which exploits the diversity and specificity of antigen binding by immunoglobulins. In such assays, monoclonal antibodies or their antigen binding domains, or polyclonal antisera (population of immunoglobulins, are used alone (e.g. immunohistochemistry) or in combination (e.g. sandwich immunoassay) to specifically bind target protein or peptide of interest. Such assays have been developed in combination with a wide range of labeling or signal enhancement strategies to allow detection of target molecules. These include fluorescent, luminescent, colorimetric, histochemical, magnetic, radioactive, and photon scattering properties, or through change in density or mass. Assay platform using these strategies include enzyme-linked immunosorbent assays (ELISA and immunospot), flow cytometry, immunohistochemistry and immunofluorescence imaging, as well as multiplexed immunoassay platforms utilizing bead, chip, and gel substrates including lateral flow immunochromatography (e.g. pregnancy test), protein array (e.g. planar glass or silicon array), flow cytometrix microbead (e.g. Luminex), and two-dimensional (e.g. paper-based capture and signal detection) or three-dimensional matrix (e.g. hydrogel).

Hence, it should be understood that there are many methods of gene product (e.g., protein and peptide, and/or RNA) quantification and detection that may be used by a platform in accordance with the methods disclosed herein.

### 4. Classification and Medical Methods

The gene markers and trained machine learning methods described herein are useful for various medical applications including the treatment and diagnosis of an Aspergillus infection. The methods provided herein may be used to analyze acquired gene expression level data from a subject to generate an output of diagnosis of the subject, i.e., whether the subject has an Aspergillus infection and to treat the subject accordingly. For example, the methods provided herein may be used to generate the diagnosis of the subject having an Aspergillus infection and administer the subject an antifungal treatment if the subject has been identified of having an Aspergillus infection.

Accordingly, provided herein is a method of identifying and/or treating an Aspergillus infection in a subject. In some instances the method comprises the steps of (a) measuring, the gene expression levels of two or more aspergillosis versus reference (“AvR”) genes selected from the group consisting of gene markers listed in Table 1 or Table 2 in a biological sample obtained from the subject, and (b) administering an effective amount of an antifungal treatment to the subject identified as having an Aspergillus infection based on comparison of the gene expression levels of the two or more AvR genes with reference gene expression levels of the AvR genes in a reference sample having a known Aspergillus infection classification. In certain embodiments, the Aspergillus infection is an Aspergillus fumigatus infection, i.e., an infection caused by Aspergillus fumigatus.

In some instances, the method comprises the steps of (a) selecting a subject who has been classified as having an Aspergillus infection based on the gene expression levels of two or more AvR genes selected from the group consisting of gene markers listed in Table 1 or Table 2 relative to reference expression levels determined for the AvR genes in a reference sample having a known Aspergillus infection classification, and (b) administering to the subject an effective amount of an antifungal treatment.

In some instances, the method comprises the steps of (a) measuring the gene expression levels of two or more AvR gene markers in a biological sample obtained from the subject, wherein the plurality of the AvR genes are selected from the group consisting of genes shown in Table 1 or Table 2, (b) using the gene expression levels of each of the AvR genes as features for a machine learning model to generate a score, wherein the gene expression levels of the AvR genes in the biological sample are compared to reference gene expression levels, wherein the reference gene expression levels are the gene expression levels for the AvR genes in a subject that does not have an Aspergillus infection, (d) identifying the subject as having an Aspergillus infection if the score is higher than the score of a subject that does not have an Aspergillus infection, and (e) administering an effective amount of an antifungal treatment to the subject identified as having an Aspergillus infection in (d).

In some instances, the method comprises the steps of (a) measuring the gene expression levels of two or more AvR genes in a biological sample obtained from the subject, wherein the plurality of the AvR genes are selected from the group consisting of gene markers shown in Table 1 or Table 2, (b) detecting potential differences in the gene expression levels of the AvR genes relative to reference gene expression levels characteristic of a subject who does not have an Aspergillus infection, (c) determining whether the subject has an Aspergillus infection based on the differences, if any, detected in (b), and (d) administering an effective amount of an antifungal treatment to the subject if the subject has been determined to have an Aspergillus infection in (c). In such cases, the reference sample is a sample obtained from a healthy subject, i.e., a subject who is not infected with Aspergillus.

In some instances, the method comprises the steps of (a) measuring, the gene expression levels of two or more AvR genes selected from the group consisting of gene markers listed in Table 1 or Table 2 in a biological sample obtained from the subject, (b) determining a classification of whether the subject has an Aspergillus infection using the gene expression levels of two or more AvR genes and reference gene expression levels of two or more AvR genes determined from reference samples having a known Aspergillus infection classification, wherein determining the classification includes inputting the gene expression levels of two or more AvR genes to a machine learning model that discriminates between different Aspergillus infection classifications, and wherein the machine learning model is trained using two or more reference gene expression levels and the known Aspergillus infection classifications of reference samples, and (d) administering an effective amount of an antifungal treatment to the subject determined to have an Aspergillus infection in (c).

In some instances, the method comprises the steps of (a) measuring the gene expression levels of two or more AvR genes in a biological sample obtained from the subject, wherein the plurality of the AvR genes are selected from the group consisting of gene markers shown in Table 1 or Table 2, (b) normalizing the gene expression levels of AvR genes to generate normalized gene expression levels, (c) inputting the normalized gene expression values into a classifier that discriminates between an Aspergillus infection classification and a uninfected classification, wherein the classifier comprises pre-defined weighting values for each of the AvR genes, (d) calculating a probability for an Aspergillus infection based on the normalized gene expression values, to thereby determine if the subject has an Aspergillus infection, and (e) administering an effective amount of an antifungal treatment to the subject if the subject has been determined to have an Aspergillus infection in (d).

Another aspect of the present disclosure provides a method of treating a fungal infection/illness whose etiology is unknown in a subject, said method comprising, consisting of, or consisting essentially of (a) obtaining a biological sample from the subject; (b) determining the gene product (e.g., protein and/or peptide) expression profile of the subject from the biological sample by evaluating the expression levels of pre-defined sets of gene products; (c) normalizing gene product (e.g., protein and/or peptide) expression levels as required for the technology used to make said measurement to generate a normalized value; (d) entering the normalized values into a fungal classifier (i.e., predictors) that have pre-defined weighting values (coefficients) for each of the gene products in each signature; (e) comparing the output of the classifiers to pre-defined thresholds, cut-off values, or ranges of values that indicate infection and/or likelihood of infection; (f) classifying the presence or absence of fungal etiology of the infection; and (g) administering to the subject an appropriate treatment regimen as identified by step (f). In some embodiments, step (g) comprises administering an antifungal therapy. In some embodiments, step (f) further comprises identifying whether the fungal infection is caused by Aspergillus if the presence of fungal etiology is found.

If the infection is found to be caused by a fungal infection, the subject may undergo treatment, for example anti-fungal therapy, and/or she may be quarantined to her home or healthcare facility for the course of the infection.

Further provided herein is a method of determining the presence of an Aspergillus infection in a subject. In some instances the method comprises (a) measuring, the gene expression levels of two or more AvR genes selected from the group consisting of gene markers listed in Table 1 or Table 2 in a biological sample obtained from the subject, and (b) identifying the subject as having an Aspergillus infection based on comparison of the gene expression levels of the two or more AvR genes in the biological sample to reference expression levels determined for the AvR genes in a reference sample having a known Aspergillus infection classification. In certain embodiments, the Aspergillus infection is an Aspergillus fumigatus infection, i.e., an infection caused by Aspergillus fumigatus.

In some instances, the method comprises the steps of (a) measuring the gene expression levels of two or more of AvR gene markers in a biological sample obtained from the subject, wherein the plurality of the AvR genes are selected from the group consisting of genes shown in Table 1 or Table 2, (b) using the gene expression levels of each of the AvR genes as features for a machine learning model to generate a score, wherein the gene expression levels of the AvR genes in the biological sample are compared to reference gene expression levels, wherein the reference gene expression levels are the gene expression levels for the AvR genes in a subject that does not have an Aspergillus infection, (c) identifying the subject as having an Aspergillus infection if the score is higher than the score of a subject that does not have an Aspergillus infection.

In some instances, the method comprises the steps of (a) measuring the gene expression levels of two or more of AvR genes in a biological sample obtained from the subject, wherein the plurality of the AvR genes are selected from the group consisting of gene markers shown in Table 1 or Table 2, (b) detecting potential differences in the gene expression levels of the AvR genes relative to reference gene expression levels characteristic of a subject who does not have an Aspergillus infection, (c) determining whether the subject has an Aspergillus infection based on the differences, if any, detected in (b).

In some instances, the method comprises the steps of (a) measuring, the gene expression levels of two or more AvR genes selected from the group consisting of gene markers listed in Table 2 in a biological sample obtained from the subject, (b) determining a classification of whether the subject has an Aspergillus infection using the gene expression levels of two or more AvR genes and reference gene expression levels of two or more AvR genes determined from reference samples having a known Aspergillus infection classification, wherein determining the classification includes inputting the gene expression levels of two or more AvR genes to a machine learning model that discriminates between different Aspergillus infection classifications, and wherein the machine learning model is trained using two or more reference gene expression levels and the known Aspergillus infection classifications of reference samples.

In some instances, the method comprises the steps of (a) measuring the gene expression levels of two or more AvR genes in a biological sample obtained from the subject, wherein the plurality of the AvR genes are selected from the group consisting of gene markers shown in Table 1 or Table 2, (b) normalizing the gene expression levels of AvR genes to generate normalized gene expression levels, (c) inputting the normalized gene expression values into a classifier that discriminates between an Aspergillus infection classification and a uninfected classification, wherein the classifier comprises pre-defined weighting values for each of the AvR genes, (d) calculating a probability for an Aspergillus infection based on the normalized gene expression values, to thereby determine if the subject has an Aspergillus infection.

Another aspect of the present disclosure provides methods for determining whether a patient has a respiratory illness due to a fungal infection (e.g., an infection caused by Aspergillus). The method for making this determination relies upon the use of classifiers obtained as taught herein. Such methods may include: a) measuring the expression levels of pre-defined sets of gene products (e.g., as set forth in Table 1 or 2); b) normalizing expression levels for the technology used to make said measurement; c) taking those values and entering them into a fungal classifier that has predefined weighting values (coefficients) for each of the gene products in each signature; d) comparing the output of the classifier to pre-defined thresholds, cut-off values, confidence intervals or ranges of values that indicate likelihood of infection; and optionally e) jointly reporting the results of the classifiers.

In some embodiments, these signatures are derived using carefully adjudicated groups of patient samples with the condition(s) of interest. After obtaining a biological sample from the patient, in some embodiments the gene product is extracted. The gene product is quantified for all, or a subset, of the genes in the signatures (e.g., those forth in Table 1 or 2). Depending upon the apparatus that is used for quantification, the gene product(s) may have to be first purified from the sample.

The signature is reflective of a clinical state. For example, the fungal infection signature is defined by a group of biomarkers (host gene markers) that distinguish patients with a fungal infection (e.g., an Aspergillus infection) from those without a fungal infection (e.g., those set forth in Table 1 or 2). Further, the fungal signature is defined by a group of biomarkers (host gene markers) that help determine the etiology of the fungal infection (e.g., caused by Aspergillus or another type of fungus) (e.g., those set forth in Table 1 or 2).

Another aspect of the present disclosure provides a method for determining the etiology of fungal infection in a subject suffering therefrom, or at risk of thereof, comprising, consisting of, or consisting essentially of: (a) obtaining a biological sample from the subject; (b) measuring on a platform the gene product expression levels of a pre-defined set of gene products (i.e., signature) in said biological sample (e.g., as set forth in Table 1 or 2); (c) normalizing the gene product expression levels to generate normalized gene product values; (d) entering the normalized gene product expression values into one or more fungal classifiers, said classifier(s) comprising pre-defined weighting values (i.e., coefficients) for each of the genes of the pre-determined set of gene products for the platform, optionally wherein said classifier(s) are retrieved from one or more databases; and (e) calculating an etiology probability for one or more of a fungal illness based upon said normalized gene products expression values and said classifier(s), to thereby determine the etiology of the fungal in the subject. In some embodiments, the determination is to identify the genus Aspergillus. In certain embodiments, the determination is to identify Aspergillus fumigatus.

Classification is the activity of assigning an observation or a patient to one or more categories or outcomes (e.g. a patient is infected with an Aspergillus or is not infected, another categorization may be that a patient is infected with a fungus that is not Aspergillus). In some cases, an observation or a patient may be classified to more than one category, e.g. in case of co-infection. The outcome, or category, is determined by the value of the scores provided by the classifier, when such predicted values are compared to a cut-off or threshold value or limit. In other scenarios, the probability of belonging to a particular category may be given if the classifier reports probabilities. In some cases, a “+” symbol (or the word “positive”) signifies that a sample is classified as having an Aspergillus infection. The classification can be binary (e.g., positive or negative) or have more levels of classification (e.g., a scale from 1 to 10 or 0 to 1). The terms “cutoff” and “threshold” refer to predetermined numbers used in an operation. A threshold value may be a value above or below of which a particular classification applies. Either of these terms can be used in either of these contexts. A cutoff or threshold may be “a reference value” or derived from a reference value that is representative of a particular classification or discriminates between two or more classifications.

The term “reference sample,” as used herein, refers to a sample having a known state (e.g., Aspergillus infection classification). Gene expression in the reference sample may be used as a baseline or reference value with which to compare expression in a test sample. In particular, the expression level of a gene from the reference sample (referred to herein as “reference gene expression level”) can be used to compare against the sample for which a classification is to be determined. In various examples, reference gene expression levels from a training set of reference samples may be used to generate a diagnostic classifier. Accordingly, a reference sample is a sample having a known Aspergillus infection classification. In some cases, the reference sample having a known Aspergillus infection classification is from a subject that does not have an Aspergillus infection. Thus, a reference sample can be sample obtained from a healthy subject, i.e., a subject who is not infected with Aspergillus. In some embodiments, the reference sample having a known Aspergillus infection classification is from a subject that has an Aspergillus infection. Thus, in some embodiments, a reference sample is a sample obtained from an infected subject having an Aspergillus infection. In some embodiments, methods of determining the presence of an Aspergillus infection in a subject involves using values obtained from reference subjects who are age and/or gender matched with the subject.

As used herein, the term biological sample comprises any sample that may be taken or obtained from a subject that contains gene product material that can be used in the methods provided herein. In some embodiments, a biological sample comprises nucleic acids, such as mRNA expressed by cells of the subject. In some embodiments, a biological sample comprises proteins or peptides expressed by cells of the subject. For example, a biological sample may comprise a nasopharyngeal lavage or wash sample or a nasal swab. Other samples may comprise those taken from the upper respiratory tract, including but not limited to, sputum, nasopharyngeal swab, respiratory expectorate, epithelial cells or tissue from upper respiratory tract. A biological sample may also comprise those samples taken from the lower respiratory tract, including but not limited to, bronchoalveolar lavage and endotracheal aspirate. A biological sample may also be blood (e.g., peripheral blood), serum, or plasma. In some embodiments, a biological sample may comprise peripheral blood cells. Additionally, a biological sample may comprise a solid tissue; for example, lung tissue (e.g., biopsy) may be used as biological samples. A biological sample may also comprise any combinations thereof.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, mouse, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject is a human who is at risk of contracting, or suffering from, a fungal infection (e.g., Aspergillus fumigatus infection).

In some aspects, the subject is suspected of having a fungal infection. In some aspects, the subject is suspected of having an Aspergillus infection. In some aspects, the subject has acute respiratory illness symptoms. In some cases, the subject has symptoms of a fungal infection. In some cases, the subject has symptoms of an Aspergillus infection. In some cases, the subject has symptoms of aspergillosis. For instance, the subject may present with symptoms such as fever, coughing, chest pain, or difficulty breathing. In some cases, if the infection has spread to other parts of the body (e.g., the ears and/or the sinuses), the subject may present with other symptoms, such as congestion, or pain in the ears or sinuses. In some cases, an examination of the subject is performed to evaluate the presence of clinical symptoms of an acute respiratory illness and/or an Aspergillus infection. In some embodiments, the examination may include a chest x-ray, computed tomography (CT) of the chest.

In some instances, the provided methods can be used to determine the presence of an Aspergillus infection in subjects who are immunocompromised and thus have an increased risk for a fungal infection (e.g., an Aspergillus infection). As described in the Examples of this disclosure, AvR genes of classifier 2 have been found to be particularly predictive of an Aspergillus infection across different immunosuppressive states. Thus, for immunocompromised subject, the step of measuring gene expression levels of AvR genes may involve measuring, the gene expression levels of two or more AvR genes selected from the group consisting of gene markers listed in Table 2.

As used herein, the term “immunocompromised” refers to a subject whose immune system is impaired or weakened and/or functioning abnormally as compared with a healthy subject. Thus, an immunocompromised subject is a subject with reduced ability to elicit an appropriate immune response against invading pathogens. An immunocompromised individual may exhibit one or more types of impairment of the immune system, such as immunosuppression, immunodeficiency, altered or overactive immune system, autoimmunity, or any combination thereof. An immunocompromised state in a subject can be due to a variety of causes, including but not limited to medications and therapies that cause immunosuppression (e.g., steroids, chemotherapy, radiation therapy, other immunosuppressive treatment), genetic disorders of the immune system, diseases, disorders and/or infections that affect the immune system (e.g., human immunodeficiency virus infection and other viral, parasitic and bacterial infections), autoimmune disorders, cystic fibrosis, sepsis, cancer, kidney failure, alcoholism, cirrhosis, diabetes, and old age. In some embodiments, an immunocompromised subject may have neutropenia.

The term “neutropenia” as used herein, refers to abnormally low concentrations of neutrophils in the blood. Neutropenia may be due to decreased production or destruction of white blood cells (for example, due to immunosuppressive agents, chemotherapy, therapeutic agents that affect the bone marrow, hereditary/congenital disorders that affect the bone marrow, aplastic anemia, cancer, radiation therapy, acute bacterial infections, certain autoimmune diseases, Vitamin B12, folate or copper deficiency and/or exposure to pesticides). The diagnosis of neutropenia may be done via the low neutrophil count detection on a complete blood count and may include bone marrow biopsy, serial neutrophil counts, and tests for antineutrophil antibodies. Reference range for absolute neutrophil count (ANC) in adults is 1500 to 8000 cells per microliter (μl) of blood. Three general guidelines are used to classify the severity of neutropenia based on the ANC (expressed below in cells/μl): Mild neutropenia (1000<=ANC<1500): minimal risk of infection; Moderate neutropenia (500<=ANC<1000): moderate risk of infection; Severe neutropenia (ANC<500): severe risk of infection.

As used herein, the term “immunosuppressive treatment” refers to any agent, medication, and/or therapy that can lead to an immunocompromised state in the subject. Immunosuppressive treatment are generally used to prevent transplant rejections, and to treat cancers, autoimmune diseases (e.g., rheumatoid arthritis, lupus, and inflammatory bowel disease), multiple sclerosis and other conditions. Immunosuppressive treatments include but are not limited to treatments that involve steroids, chemotherapy (exemplary agents are described below), radiation therapy, Azathioprine, Mycophenylate mofetil (MMF), Cyclosporine, Tacrolimus, Sirolimus, Anti-thymocyte globulin (ATG), Alemtuzumab, Rituximab, Basiliximab, Belatacept, Bortezomib, Eculizumab.

In some instances, the provided methods may be used to identify early signs of an Aspergillus infection in immunocompromised subjects. In some instances the subject receives or has previously received a treatment or therapy that causes immunosuppression. In some instances, the subject is monitored during the course of or after an immunosuppressive treatment (e.g., chemotherapy or steroids) or therapy using the provided methods. For example, the methods provided herein may be used for early detection and/or treatment of an Aspergillus infection before the manifestation of clinical symptoms. In some cases, confirmation of an Aspergillus infection with the provided methods may allow targeted treatment of the condition (e.g., with an antifungal treatment; described further below) while allowing the subject to continue to be treated with chemotherapy or steroids.

In some instances, the subject has received an organ transplantation. For example, the subject may have received a solid organ transplantation, such as a lung, heart, heart valve, liver, kidney, or a pancreas transplantation. In some aspects, the subject has received a lung transplantation.

In other instances, the subject has received a stem cell transplantation (also known as bone marrow transplantation). Stem cell transplants are used to treat hematological malignancies, including various types of leukemias, lymphomas, or myelomas. In some cases, the subject has received a hematopoietic stem cell transplantation. In such cases, the subject may have received high doses of steroids (e.g., glucocorticoids such as prednisone) in order to prevent or treat graft-versus-host disease (GVHD). Accordingly, in some aspects, the provided methods of diagnosing and/or treating an Aspergillus infection may be used in subjects who have received a stem cell transplantation and/or have been treated with high doses of steroids. In some cases, the provided methods are useful for monitoring a subject who has received a stem cell transplantation and is being treated or has previously been treated with steroids to detect early evidence of an Aspergillus infection.

In another instance, the subject may be a patient who is being treated or has previously been treated with chemotherapy or other anti-cancer treatments that cause immunosuppression. For example, the subjects may have or have had cancer (e.g., leukemia) and may receive or have previously received chemotherapy for treating their cancer. For some cases, a subject that is treated or has previously been treated with chemotherapy may develop signs and symptoms associated with a fungal infection (e.g., an Aspergillus infection). In some cases, a subject that receives or has previously received chemotherapy may develop signs and symptoms associated with an acute respiratory illness. In some instances, the provided methods are useful for investigating and treating acute respiratory illness symptoms that appear during or after chemotherapy and are not explainable by the cancer.

As used herein, the term “chemotherapy” refers to the treatment of cancer using specific chemical agents or drugs that are destructive of malignant cells and tissues. Various types of chemotherapies exist, such as alkylating agents, antitumor antibiotics, antimetabolites, topoisomerase inhibitors or the like. Exemplary chemical agents that are used, include, but are not limited to, cytarabine, fludarabine, azacitidine, venetoclex, clofarabine, decitabine, busulfan, ibrutinib, cyclophosphamide, docetaxel, hydroxydaunorubicin, adriamycin, doxorubicin, vincristine, prednisone, prednisolone, and temozolomide.

In some aspects, the subject is an immunocompromised patient that is receiving or has previously received immunosuppressive treatments (e.g., chemotherapy, steroids etc.) and thus is at risk of developing fungal infection (e.g., an Aspergillus infection) but cannot prophylactically be treated with antifungal medication. For example, some patients may have received prolonged antifungal treatment previously and may be at risk of developing resistance to antifungal treatment. Thus, prophylactic antifungal treatment may not be appropriate in such patients. In such cases, the provided methods are useful for monitoring and screening these patients to identify a potential Aspergillus infection. The patients can then be administered with antifungal treatment only as needed if they have been diagnosed to have an Aspergillus infection. In some cases, these patients may present with any signs or symptoms associated with an Aspergillus infection or an acute respiratory illness.

In some instances, the subject is an individual that has previously had a viral acute respiratory illness. These patients may be immunocompromised due to the viral infection and/or their respiratory system may be weakened and thus be more prone to a fungal infection (e.g., an Aspergillus infection). For example, the subject may have had a viral acute respiratory illness, such as influenza (e.g., influenza caused by a type A, type B, type C, or type D influenza virus), coronavirus disease 2019 (COVID-19) caused by the severe acute respiratory syndrome coronavirus 2 (SARS-CoV-2) or an respiratory illnesses caused by other coronaviruses (e.g., SARS-CoV, middle eastern respiratory syndrome (MERS) virus), or respiratory illnesses caused by paramyxoviridae, picornaviruses, or adenoviruses.

In some instances, the subject is an individual that having a lung disease. Lung diseases that may increase the risk for aspergillosis include but are not limited to chronic obstructive pulmonary disease (COPD), asthma, emphysema, bronchitis. Lung cancer, pneumonia, pulmonary edema, pulmonary embolus, pneumoconiosis, and pneumothorax.

In other cases, the subject is a healthy and/or immunocompetent individual. In such cases, gene expression levels of two or more genes as listed in Table 1 or Table 2 can be used to determine the presence of an Aspergillus infection.

In some embodiments, the subject receives an appropriate treatment regimen (e.g., an antifungal treatment) to treat the Aspergillus infection. In some embodiments, the subject is administered an effective amount of an antifungal treatment.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In the context of a fungal infection, such terms can also refer to a reduction in the replication of the fungus, or a reduction in the spread of the fungus to other organs or tissues in a subject or to other subjects. Treatment may also include therapies for non-infectious illnesses, such as allergy treatment, asthma treatments, and the like.

The term “appropriate treatment regimen” refers to the standard of care needed to treat a specific disease or disorder. Often such regimens require the act of administering to a subject a therapeutic agent(s) capable of producing a curative effect in a disease state. For example, a therapeutic agents for treating a subject having a fungal infection (e.g., an Aspergillus infection) include, but are not limited to, itraconazole, voriconazole, lipid amphotericin formulations (e.g., amphotericin B), posaconazole, isavuconazole, caspofungin, micafungin, anidulafungin, and other antifungal medications. Such a treatment may also be referred to as an “antifungal treatment”. The present disclosure contemplates the use of the methods of the present disclosure to determine treatments with antifungal medications and/or other antifungal treatments that are not yet available.

The term “effective amount” or “therapeutically effective amount” refers to an amount sufficient to effect beneficial or desirable biological and/or clinical results. A desirable biological and/or clinical result result would include an improvement in a symptom associated with a fungal infection, particularly a respiratory fungal infection. A therapeutically effective amount of such a composition may vary according to factors such as the disease state, age, sex, and weight of the individual. Dosage regimens may be adjusted to provide the optimum response. A therapeutically effective amount is also one in which any toxic or detrimental effects of the antifungal treatment are outweighed by the therapeutically beneficial effects.

As used herein, the term “administering”, “administration”, or “administer” means delivering a pharmaceutical composition (e.g., an antifungal medication) to a subject. The antifungal medication may be delivered to subjects in need thereof by any suitable route or a combination of different routes. In particular embodiments, antifungal medications are administered orally, intravenously, or inhaled.

The person performing the biological sample procurement need not perform the comparison, however, as it is contemplated that a laboratory may communicate the gene product (e.g., protein and/or peptide) classification results to a medical practitioner for the purpose of identifying the etiology of the infection (e.g., whether the infection is caused by a fungus [e.g., an Aspergillus infection]) and for the administration of appropriate treatment. Additionally, it is contemplated that a medical professional, after examining a patient, would order an agent to obtain a biological sample, have the sample assayed for the classifiers as provided herein, and have the agent report patient's fungal etiological status to the medical professional. Once the medical professional has obtained the classification result, the medical professional could order suitable treatment and/or quarantine.

The methods provided herein can be effectively used to determine the presence or absence of a fungal infection in order to correctly treat the patient and reduce inappropriate use of antibiotics or other non-effective treatments. Further, the methods provided herein have a variety of other uses, including but not limited to, (1) a host-based test to detect individuals who have been exposed to a pathogen and have impending, but not symptomatic, illness (e.g., in scenarios of natural spread of diseases through a population); (2) a host-based test for monitoring response to a vaccine or a drug, either in a clinical trial setting or for population monitoring of immunity; (3) a host-based test for screening for impending illness prior to deployment (e.g., a military deployment or on a civilian scenario such as embarkation on a cruise ship); and (4) a host-based test for the screening of livestock for fungal infections.

The methods described herein are also useful for practitioners to help determine when an antibiotic should or should not be prescribed to a subject suffering from an acute respiratory infection. Overuse and misapplication of antibacterial therapies have led to unfortunate consequences, such as the development of antibiotic resistant strains of bacteria.

### 5. Methods of Generating Classifiers (Training)

The present disclosure provides methods of generating a classifier(s) (also referred to as training) for use in the methods of determining the presence or absence of a fungal infection (e.g., Aspergillus infection) in a subject. In other aspects, the present disclosure provides methods for determining the etiology of a fungal infection in a subject. Gene, protein, or peptide expression-based classifiers have been developed that can be used to identify and characterize the presence of and/or absence of a fungal infection in a subject with a high degree of accuracy. In some embodiments, the fungal infection is an Aspergillus infection. In some instances, the Aspergillus infection is an A. fumigatus infection.

As used herein, the terms “classifier” and “predictor” are used interchangeably and refer to a mathematical function that uses the values of the signature (e.g. gene expression levels or protein and/or peptide levels from a defined set of gene products) and a pre-determined coefficient for each signature component to generate scores for a given observation or individual patient for the purpose of assignment to a category. A classifier is linear if scores are a function of summed signature values weighted by a set of coefficients. Furthermore, a classifier is probabilistic if the function of signature values generates a probability, a value between 0 and 1.0 (or 0 and 100%) quantifying the likelihood that a subject or observation belongs to a particular category or will have a particular outcome, respectively. Probit regression and logistic regression are examples of probabilistic linear classifiers.

A classifier, including a linear classifier, may be obtained by a procedure known as training, which consists of using a set of data containing observations with known category membership (see Example 1). Specifically, training seeks to find the optimal coefficient for each component of a given signature, where the optimal result is determined by the highest classification accuracy. In some embodiments, a unique classifier may be developed and trained with respect to a particular platform upon which the signature is measured.

For example, classifiers that use host gene expression levels can be generated from a training set of samples obtained from patients having a known Aspergillus infection classifications, e.g., for diagnosis. Measurements of many host genes can be obtained. The measurements can be analyzed to determine set of genes (i.e., their expression levels) that best discriminate between the different classifications of the training set via an optimization procedure. The analysis of gene expression data can include training a machine learning model to distinguish between positive and negative samples based on the expression level of certain genes. The analysis can include using the gene expression data as a training set where the gene expression levels and known diagnosis are used to train a machine learning model to distinguish between positive and negative samples. In the process of learning, the model identifies gene markers that are predictive for the Aspergillus infection.

Hence, one aspect of the present disclosure provides a method of making a fungal infection classifier comprising, consisting of, or consisting essentially of (i) obtaining a biological sample from a plurality of subjects suffering from a fungal infection; (ii) processing the gene product fraction from the biological sample (e.g., isolating mRNA, proteins, and/or peptides from said sample to create an expressed transcriptome or proteome); (iii) measuring the expression levels of a plurality of the gene products (e.g., mRNA, proteins, and/or peptides) (i.e., some or all of the gene products expressed in the transcriptome and/or proteome); normalizing the expression levels; generating a fungal infection classifier to include normalized gene product (e.g., peptide and/or protein) expression levels and a “weighting” coefficient value; and optionally, (vi) uploading the classifier (e.g., peptide identity and weighing coefficient) to a database. In some embodiments, the fungal infection comprises an infection with the fungus Aspergillus.

In some embodiments, the sample is not purified after collection. In some embodiments, the sample may be purified to remove extraneous material, before or after lysis of cells. In some embodiments, the sample is purified with cell lysis and removal of cellular materials, isolation of nucleic acids, and/or reduction of abundant transcripts such as globin or ribosomal RNAs.

In some embodiments, the method further includes uploading the final gene product target list for the generated classifier, the associated weights (wₙ), and threshold values to one or more databases.

The methodology for training described herein may be readily translated by one of ordinary skill in the art to different gene product expression detection platforms (e.g., mRNA and/or protein/peptide detection and quantification).

The methods and assays of the present disclosure may be based upon gene products expression, for example, through direct measurement of mRNA or proteins, measurement of derived or component materials (e.g., peptides), and measurement of other products (e.g., metabolites). In some embodiments, the gene expression level may be determined by measuring mRNA. Any method of extracting and screening gene product expression may be used and is within the scope of the present disclosure.

In some embodiments, the measuring comprises the detection and quantification (e.g., semi-quantification) of the gene products in the sample. In some embodiments, the gene product expression levels are adjusted relative to one or more standard level(s) (“normalized”). As known in the art, normalizing is done to remove technical variability inherent to a platform to give a quantity or relative quantity (e.g., of expressed genes).

In some embodiments, the measurement of differential expression of specific gene products from biological samples may be accomplished using a range of technologies, reagents, and methods. These include any of the methods of measurement as described above in Section 3.

The expression levels are typically normalized following detection and quantification as appropriate for the particular platform using methods routinely practiced by those of ordinary skill in the art.

With gene product detection and quantification and a matched normalization methodology in place for platform, it is simply a matter of using carefully selected and adjudicated patient samples for the training methods. For example, the cohort described herein was used to generate the appropriate weighting values (coefficients) to be used in conjunction with the gene product in the signature for a platform. These subject-samples could also be used to generate coefficients and cut-offs for a test implemented using a different gene products detection and quantification platform.

In some embodiments, the signatures may be obtained using a supervised statistical approach known as sparse linear classification in which sets of gene products are identified by the model according to their ability to separate phenotypes during a training process that uses the selected set of patient samples. The outcomes of training is a gene product signature(s) and classification coefficients for the classification comparison. Together the signature(s) and coefficient(s) provide a classifier or predictor. Training may also be used to establish threshold or cut-off values.

Threshold or cut-off values can be adjusted to change test performance, e.g., test sensitivity and specificity. For example, the threshold for a fungal infection may be intentionally lowered to increase the sensitivity of the test for fungal infection, if desired.

In some embodiments, the classifier generating comprises iteratively: (i) assigning a weight for each normalized gene product expression value, entering the weight and expression value for each gene product into a classifier (e.g., a linear regression classifier) equation and determining a score for outcome for each of the plurality of subjects, then (ii) determining the accuracy of classification for each outcome across the plurality of subjects, and then (iii) adjusting the weight until accuracy of classification is optimized. Gene products having a non-zero weight are included in the respective classifier.

Determining the accuracy of classification may involve the use of accuracy measures such as sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting a fungal infection (e.g., an Aspergillus infection).

In some embodiments, the classifier is a linear regression classifier and said generating comprises converting a score of said classifier to a probability using a link function. As known in the art, the link function specifies the link between the target/output of the model (e.g., probability of fungal infection) and systematic components (in this instance, the combination of explanatory variables that comprise the predictor) of the linear model. It says how the expected value of the response relates to the linear predictor of explanatory variable.

In some embodiments, the classifiers that are developed during training and using a training set of samples are applied for prediction purposes to diagnose new individuals (“classification”). For each subject or patient, a biological sample is taken and the normalized levels of expression (i.e., the relative amount of gene product expression) in the sample of each of the gene products specified by the signatures found during training are the input for the classifier. In other embodiments, the classifier can also use the weighting coefficients discovered during training for each gene product. As outputs, the classifiers are used to compute probability values. Each probability value may be used to determine the presence or absence of a fungus (e.g., Aspergillus) infecting, or likely to infect, the subject.

In some embodiments, these values may be reported relative to a reference range that indicates the confidence with which the classification is made. In some embodiments, the output of the classifier may be compared to a threshold value, for example, to report a “positive” in the case that the classifier score or probability exceeds the threshold indicating the presence of a fungus. If the classifier score or probability fails to reach the threshold, the result would be reported as “negative” for the respective condition.

It should be noted that a classifier obtained with one platform may not show optimal performance on another platform. This could be due to the promiscuity of probes or other technical issues particular to the platform. Accordingly, also described herein are methods to adapt a signature as taught herein from one platform for another.

### 6. Kits and Computer Systems

### Kits

Also provided herein are kits for determining the presence of an Aspergillus infection in a subject. In some instances, the kit comprises (a) a means for extracting a biological sample; (b) a means for generating one or more arrays consisting of a plurality of synthetic oligonucleotides with regions homologous to a group of genes as listed in Table 1 and/or Table 2; and (c) instructions for use (e.g., including use of the classifiers and gene signatures described in this disclosure, values for reference gene expression levels, and directions for interpreting results based on scores and probability values).

Another aspect of the present disclosure provides a method of using a kit for assessing a classifier comprising (a) generating one or more arrays consisting of a plurality of synthetic oligonucleotides with regions homologous to a group of gene as listed in Table 1 and/or Table 2; (b) adding to said array oligonucleotides with regions homologous to normalizing genes; (c) obtaining a biological sample from a subject suspected to have an Aspergillus infection; (d) isolating RNA from said sample to create a transcriptome; (e) measuring said transcriptome on said array; (f) normalizing the measurements of said transcriptome to the normalizing genes, electronically transferring normalized measurements to a computer to implement the classifier algorithm(s), (g) generating a report; and optionally (h) administering an appropriate treatment based on the results.

Yet another aspect of the present disclosure provides a kit comprising one or more polynucleotides for specifically hybridizing to at least a section of one or more genes listed in Table 1 or Table 2. In one embodiment, the kit includes one or more polynucleotides for specifically hybridizing to at least a section of one or more genes listed in Table 1 or Table 2 for use in testing a subject for an Aspergillus infection. In one aspect, provided herein is a medical or diagnostic device that can, for example, measure gene expression levels and provide a color indication when the gene marker(s) of interest shows differential gene expression levels in a subject. The device could be used in a clinical setting to determine if a subject has an Aspergillus infection.

In some embodiments, a kit or a panel as provided herein includes a reference sample, such as a sample from a healthy subject not infected with Aspergillus. In some embodiments, a kit or a panel as provided herein includes a reference sample, such as a sample from an infected subject having an Aspergillus infection. If such a sample is included, the measurement values (reference gene expression levels) for such sample are compared with the results of the test sample, so that the presence or absence of an Aspergillus infection in the subject can be determined.

In another aspect, provided in this disclosure is a kit for determining the presence or absence of fungal etiology (e.g., an Aspergillus infection) of an acute respiratory illness in a subject comprising, consisting of, or consisting essentially of (a) a means for extracting a biological sample; (b) a means for generating one or more arrays or assay panels consisting of a plurality of antibodies, antibody fragments, aptamers, or other analyte specific or signal-generating reagents (e.g. labeled secondary antibody) for use in measuring gene product expression levels as taught herein; and (c) instructions for use.

Another aspect of the present disclosure provides a kit for determining the presence or absence of fungal etiology of an acute respiratory illness in a subject comprising, consisting of, or consisting essentially of (a) a means for extracting a biological sample; (b) a means for measuring expression levels of one or more gene products consisting of “spike-in” labeled peptides or protein fragments (e.g. stable isotope labeled peptides) for use in relative quantitation of endogenous gene product expression levels (e.g. mass spectrometry) as taught herein; and (c) instructions for use.

Yet another aspect of the present disclosure provides a kit for detecting the presence of an Aspergillus infection in a subject comprising, consisting of, or consisting essentially of (a) a means for extracting a biological sample; (b) a means for generating one or more arrays consisting of a plurality of antibodies or other analyte specific reagents for use in measuring gene product expression levels as taught herein; and (c) instructions for use.

### Classification Systems

In some embodiments, provided is a method for determining a classification of the presence or absence for an Aspergillus infection in a subject. In some instances, the method can be performed by a classification system and/or computer program. In some embodiments, expression level data can be received at the classification system, e.g., from a detection or measuring apparatus, such as a PCR device or a sequence machine that provides data to a storage device (which can be loaded into the classification system) or across a network to the computer classification. The received data can then be analyzed, interpreted and visualized by the classification system. In some examples, the present disclosure provides systems, methods, or kits that can include data analysis realized in measurement devices (e.g., laboratory instruments, such as a PCR device or sequencing machine).

Accordingly, aspects of the present disclosure provide a classification system and/or computer program product that may be used in or by a platform, according to various embodiments described herein. A classification system and/or computer program product may be embodied as one or more enterprise, application, personal, pervasive and/or embedded computer systems that are operable to receive, transmit, process and store data using any suitable combination of software, firmware and/or hardware and that may be standalone and/or interconnected by any conventional, public and/or private, real and/or virtual, wired and/or wireless network including all or a portion of the global communication network known as the Internet, and may include various types of tangible, non-transitory computer readable medium.

The term “computer readable medium” refers to any device or system for storing and providing information (e.g., data and instructions) to a computer processor. Examples of computer readable media include, but are not limited to, DVDs, CDs hard disk drives, magnetic tape and servers for streaming media over networks, and applications, such as those found on smart phones and tablets. In various embodiments, aspects of the present disclosure including data structures and methods may be stored on a computer readable medium. Processing and data may also be performed on numerous device types, including but not limited to, desktop and lap top computers, tablets, smart phones, and the like.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

In one embodiment, the classification system may include a processor subsystem, including one or more Central Processing Units (CPU) on which one or more operating systems and/or one or more applications run. The processor(s) may be either electrically interconnected or separate. Processor(s) are configured to execute computer program code from memory devices, such as memory, to perform at least some of the operations and methods described herein, and may be any conventional or special purpose processor, including, but not limited to, digital signal processor (DSP), field programmable gate array (FPGA), application specific integrated circuit (ASIC), and multi-core processors.

The memory subsystem may include a hierarchy of memory devices such as Random Access Memory (RAM), Read-Only Memory (ROM), Erasable Programmable Readonly Memory (EPROM) or flash memory, and/or any other solid state memory devices.

A storage circuit may also be provided, which may include, for example, a portable computer diskette, a hard disk, a portable Compact Disk Read-Only Memory (CDROM), an optical storage device, a magnetic storage device and/or any other kind of disk- or tape-based storage subsystem. The storage circuit may provide non-volatile storage of data/parameters/classifiers for the classification system. The storage circuit may include disk drive and/or network store components. The storage circuit may be used to store code to be executed and/or data to be accessed by the processor. In some embodiments, the storage circuit may store databases that provide access to the data/parameters/classifiers used for the classification system such as the signatures, weights, thresholds, etc. Any combination of one or more computer readable media may be utilized by the storage circuit. The computer readable media may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. As used herein, a computer readable storage medium may be any tangible medium that can contain, or store a program for use by or in connection with an instruction execution system, apparatus, or device.

An input/output circuit may include displays and/or user input devices, such as keyboards, touch screens and/or pointing devices. Devices attached to the input/output circuit may be used to provide information to the processor by a user of the classification system. Devices attached to the input/output circuit may include networking or communication controllers, input devices (keyboard, a mouse, touch screen, etc.) and output devices (printer or display). The input/output circuit may also provide an interface to devices, such as a display and/or printer, to which results of the operations of the classification system can be communicated so as to be provided to the user of the classification system.

An optional update circuit may be included as an interface for providing updates to the classification system, Updates may include updates to the code executed by the processor that are stored in the memory and/or the storage circuit. Updates provided via the update circuit may also include updates to portions of the storage circuit related to a database and/or other data storage format which maintains information for the classification system, such as the signatures, weights, thresholds, etc.

The sample input circuit of the classification system may provide an interface for the platform as described hereinabove to receive biological samples to be analyzed. The sample input circuit may include mechanical elements, as well as electrical elements, which receive a biological sample provided by a user to the classification system and transport the biological sample within the classification system and/or platform to be processed. The sample input circuit may include a bar code reader that identifies a bar-coded container for identification of the sample and/or test order form. The sample processing circuit may further process the biological sample within the classification system and/or platform so as to prepare the biological sample for automated analysis. The sample analysis circuit may automatically analyze the processed biological sample. The sample analysis circuit may be used in measuring, e.g., gene product levels of a pre-defined set of proteins and/or peptides with the biological sample provided to the classification system. The sample analysis circuit may also generate normalized expression values by normalizing the gene product (e.g., protein and/or peptide) expression levels. The sample analysis circuit may retrieve from the storage circuit a fungal classifier as provided herein comprising pre-defined weighting values (i.e., coefficients) for each of the gene products (e.g., proteins and/or peptides) of the pre-defined set of gene products. The sample analysis circuit may enter the normalized expression values into one or more classifiers selected from the fungal classifier. The sample analysis circuit may calculate an etiology probability (e.g., whether the fungus is Aspergillus or not) for one or more of the samples based upon said classifier(s) and control output, via the input/output circuit, of a determination of presence or absence of fungal etiology of the respiratory infection, or some combination thereof.

The sample input circuit, the sample processing circuit, the sample analysis circuit, the input/output circuit, the storage circuit, and/or the update circuit may execute at least partially under the control of the one or more processors of the classification system. As used herein, executing “under the control” of the processor means that the operations performed by the sample input circuit, the sample processing circuit, the sample analysis circuit, the input/output circuit, the storage circuit, and/or the update circuit may be at least partially executed and/or directed by the processor, but does not preclude at least a portion of the operations of those components being separately electrically or mechanically automated. The processor may control the operations of the classification system, as described herein, via the execution of computer program code.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the classification system, partly on the classification system, as a stand-alone software package, partly on the classification system and partly on a remote computer or entirely on the remote computer or server. In the latter scenario, the remote computer may be connected to the classification system 1 100 through any type of network, including a local area network (LAN) or a wide area network (WAN), or the connection may be made to an external computer (for example, through the Internet using an Internet Service Provider) or in a cloud computer environment or offered as a service such as a Software as a Service (SaaS).

The classification systems described herein can be implemented in hardware, software, firmware, or combinations of hardware, software and/or firmware. In some examples, the classification systems described in this specification may be implemented using a non-transitory computer readable medium storing computer executable instructions that when executed by one or more processors of a computer cause the computer to perform operations. Computer readable media suitable for implementing the classification systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a classification system described in this specification may be located on a single device or computing platform or may be distributed across multiple devices or computing platforms.

In some embodiments, the system includes computer readable code that can transform quantitative, or semi-quantitative, detection of gene expression to a cumulative score or probability of the fungal etiology of the infection.

In some embodiments, the system is a sample-to-result system, with the components integrated such that a user can simply insert a biological sample to be tested, and some time later (preferably a short amount of time, e.g., 30 or 45 minutes, or 1, 2, or 3 hours, up to 8, 12, 24 or 48 hours) receive a result output from the system.

It is to be understood that the present disclosure is not limited in its application to the details of construction and the arrangement of components set forth in the following description or illustrated in the following drawings. The present disclosure is capable of other embodiments and of being practiced or of being carried out in various ways.

In accordance with the present disclosure, there may be employed conventional molecular biology, microbiology, biochemical, and recombinant DNA techniques within the skill of the art. Such techniques are explained fully in the literature. The provided methods will be further described in the following examples, which are provided by way of illustration and which do not limit the scope of the methods of matter described in the claims.

### 7. Exemplary Embodiments

Exemplary embodiments of the invention include:

1. A method of treating an Aspergillus infection in a subject, the method comprising: (a) measuring, the gene expression levels of two or more aspergillosis versus reference (“AvR”) genes selected from the group consisting of gene markers listed in Table 2 in a biological sample obtained from the subject; and (b) administering an effective amount of an antifungal treatment to the subject identified as having an Aspergillus infection based on comparison of the gene expression levels of the two or more AvR genes with reference gene expression levels of the AvR genes in a reference sample having a known Aspergillus infection classification.

2. A method of treating an Aspergillus infection in a subject, the method comprising: (a) selecting a subject who has been classified as having an Aspergillus infection based on the gene expression levels of two or more aspergillosis versus reference (“AvR”) genes selected from the group consisting of gene markers listed in Table 2 relative to reference expression levels determined for the AvR genes in a reference sample having a known Aspergillus infection classification; and (b) administering to the subject an effective amount of an antifungal treatment.

3. A method of determining the presence of an Aspergillus infection in a subject, the method comprising: (a) measuring, the gene expression levels of two or more aspergillosis versus reference (“AvR”) genes selected from the group consisting of gene markers listed in Table 2 in a biological sample obtained from the subject; and (b) identifying the subject as having an Aspergillus infection based on comparison of the gene expression levels of the two or more AvR genes in the biological sample to reference expression levels determined for the AvR genes in a reference sample having a known Aspergillus infection classification.

4. The method of any one of embodiments 1 to 3, wherein the gene expression levels of all the gene markers listed in Table 2 are measured.

5. The method of any one of embodiments 1 to 4, wherein the biological sample is blood, serum, plasma, lung tissue, or a sample that is obtained using a nasal swab, a nasopharyngeal swab, an oropharyngeal swab, a buccal swab, a broncho-alveolar lavage, or a tracheobronchial aspirate.

6. The method of any one of embodiments 1 to 5, wherein measuring the gene expression level(s) comprises performing polymerase chain reaction (PCR), isothermal amplification, next generation sequencing (NGS), mass spectrometry, microarray analysis, enzyme-linked immunosorbent assay (ELISA), Northern blot, or serial analysis of gene expression (SAGE).

7. The method of any one of embodiments 1 to 6, wherein the measured gene expression levels are RNA expression levels measured by polymerase chain reaction (PCR) or microarray analysis, or a combination thereof.

8. The method of any one of embodiments 1 to 7, wherein the reference sample having a known Aspergillus infection classification is from a subject that does not have an Aspergillus infection.

9. The method of any one of embodiments 1 to 7, wherein the reference sample having a known Aspergillus infection classification is from a subject that has an Aspergillus infection.

10. The method of any one of embodiments 1 to 9, wherein the subject is suspected of having a fungal infection.

11. The method of any one of embodiments 1 to 10, wherein the subject is suspected of having an Aspergillus infection.

12. The method of any one of embodiments 1 to 11, wherein the subject has acute respiratory illness symptoms.

13. The method of any one of embodiments 1 to 12, wherein the subject has symptoms of a fungal infection.

14. The method of any one of embodiments 1 to 13, wherein the subject has symptoms of an Aspergillus infection.

15. The method of any one of embodiments 1 to 14, wherein the subject is immunocompromised.

16. The method of any one of embodiments 1 to 15, wherein the subject has received an organ transplantation or stem cell transplantation.

17. The method of any one of embodiments 1 to 16, wherein the subject has been previously treated or is being treated with steroids.

18. The method of any one of embodiments 1 to 17, wherein the subject has been previously treated or is being treated with chemotherapy.

19. The method of any one of embodiments 1 to 18, wherein the subject previously has had a viral acute respiratory infection.

20. A method of generating (making) a fungal infection classifier for a platform, said method comprising: (a) obtaining biological samples from a plurality of subjects known to be suffering from a fungal infection; (b) measuring on said platform the expression levels of a plurality of pre-defined gene products in each of said biological samples from step (a); (c) normalizing the gene product expression levels obtained in step (b) to generate normalized expression values; and (d) generating a fungal classifier for the platform based upon said normalized gene product expression values, to thereby make the fungal classifier for the platform.

21. The method of embodiment 20, wherein the measuring comprises, or is preceded by, one or more steps of: purifying cells, cellular materials, or secreted materials from said sample, preserving or disrupting the cells or cellular materials of said sample, and reducing complexity of sample through isolating or fractionating gene products from said sample.

22. The method of embodiment 20 or 21, wherein the measuring comprises quantitative or semi-quantitative direct detection or indirect detection using analyte specific reagents or methods.

23. The method of embodiment 22, wherein the analyte specific reagents are selected from the group consisting of antibodies, antibody fragments, aptamers, peptides, nucleic acid probes, primers, and combinations thereof.

24. The method of any one of embodiments 20-23, wherein the platform is selected from the group consisting of an array platform, a gene product analyte hybridization or capture platform, multi-signal coded detector platform, a mass spectrometry platform, an amino acid sequencing platform, a nucleic acid sequencing platform, a PCR or other amplification platform, ELISA, Northern blot, SAGE, or a combination thereof.

25. The method of any one of embodiments 20-24, wherein the generating comprises iteratively: (i) assigning a weight for each normalized gene product expression value, entering the weight and expression value for each gene product into a classifier equation and determining a score for outcome for each of the plurality of subjects, then (ii) determining the accuracy of classification for each outcome across the plurality of subjects, and then (iii) adjusting the weight until accuracy of classification is optimized to provide said fungal infection for the platform, wherein analytes having a non-zero weight are included in the respective classifier, and optionally uploading components of each classifier (gene product analytes, weights and/or etiology threshold value) onto one or more databases.

26. The method of any one of embodiments 20-25, wherein the method further comprises validating said fungal classifier against a known dataset comprising at least two relevant clinical attributes, and optionally determining a threshold for the determination of fungal infection.

27. The method of any one of embodiments 20-26, wherein the fungal infection comprises an Aspergillus infection.

28. A method for determining the presence of a fungal infection in a subject or for determining the fungal etiology of an acute respiratory infection in a subject suffering therefrom, comprising: (a) obtaining a biological sample from the subject; (b) measuring on a platform expression levels of a pre-defined set of gene products in said biological sample; (c) normalizing the gene product expression levels to generate normalized expression values; (d) entering the normalized gene product expression values into one or more fungal classifiers, said classifier(s) comprising pre-defined weighting values for each of the gene products of the pre-determined set of proteins and/or peptides for the platform, optionally wherein said classifier(s) are retrieved from one or more databases; and (e) calculating a presence or an etiology probability for one or more of the fungal infections based upon said normalized expression values and said classifier(s), and optionally determining a threshold for the determination of a fungal infection, to thereby determine the presence of a fungal infection in the subject or the fungal etiology of a respiratory illness in the subject.

29. A method for determining whether a subject is at risk of developing a fungal infection, or for determining the presence of a latent or subclinical respiratory fungal infection in a subject exhibiting no symptoms, comprising: (a) obtaining a biological sample from the subject; (b) measuring on a platform expression levels of a pre-defined set of gene products in said biological sample; (c) normalizing the gene product expression levels to generate normalized expression values; (d) entering the normalized gene product expression values into one or more acute respiratory virus illness classifiers, said classifier(s) comprising pre-defined weighting values for each of the gene products of the pre-determined set of proteins and/or peptides for the platform, optionally wherein said classifier(s) are retrieved from one or more databases; and (e) calculating a risk probability or a probability for one or more of a fungal infection based upon said normalized expression values and said classifier(s), and optionally determining a threshold for the determination of fungal infection, to thereby determine whether the subject is a risk of developing a fungal infection, or to determine the presence of a latent respiratory fungal infection in the subject.

30. The method of embodiment 28 or 29, wherein the method further comprises: (f) comparing the probability to pre-defined thresholds, cut-off values, or ranges of values that indicate an infection or a likelihood of infection.

31. The method of any one of embodiments 28-30, wherein the gene product is selected from the group consisting of proteins, component peptides, expressed proteome, epitopes or subset thereof and combinations thereof.

32. The method of any one of embodiments 28-31, wherein the subject is suffering from acute respiratory fungal illness symptoms or the subject is suspected of having a fungal infection.

33. The method of any one of embodiments 28-32, wherein the etiology of the fungal infection comprises an Aspergillus infection.

34. The method of any one of embodiments 28-32, wherein the pre-defined set of gene products comprises from 2, 5, 8, 10, 15, 20, 25, 30, 40, 50, 60, 70, 80, 90, 100, 110, 120, 130, 140, or more gene products.

35. The method of any one of embodiments 28-34, wherein the pre-defined set of gene products comprises 2, 5, 8, 10, 15, 20, 25, 30, 35, 40, 45, 50, 55, 60, 65, 70, 75, 80, 85, 90, 95, 100, 105, 110, 115, 120, 125, 130, 135, 140, 145 or 146 gene products (e.g., RNAs, proteins, and/or component peptides) of the gene markers listed in Table 1 or 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 187 gene products of the gene markers listed in Table 2.

36. The method of any one of embodiments 28-35, wherein the biological sample is selected from the group consisting of peripheral blood, sputum, nasal or nasopharyngeal swab, nasopharyngeal lavage, bronchoalveolar lavage, endotracheal aspirate, respiratory expectorate, respiratory epithelial cells or tissue, or other respiratory cell, tissue, or secretion samples and combinations thereof.

37. The method of any one of embodiments 28-36, wherein the biologic sample is obtained as a nasal or respiratory spray captured onto paper-based matrix for extraction or direct assay.

38. A method of treating comprising administering to the subject an appropriate treatment regimen based on the fungal etiology determined by a method according to embodiment 28.

39. The method of embodiment 38, wherein the appropriate treatment regimen comprises an antifungal therapy.

40. A method of monitoring response to a vaccine, drug or other antifungal therapy in a subject suffering from, or at risk of developing, a fungal infection comprising determining a host response of said subject using a method of any one of embodiments 3, 28 or 29.

41. The method of embodiment 40, wherein the drug is an antifungal drug.

42. A system for determining the presence of and/or determining the etiology of a fungal infection in a subject comprising: (i) at least one processor; (ii) a sample input circuit configured to receive a biological sample from the subject; (iii) a sample analysis circuit coupled to the at least one processor and configured to determine gene product expression levels of the biological sample; (iv) an input/output circuit coupled to the at least one processor; (v) a storage circuit coupled to the at least one processor and configured to store data, parameters, and/or classifiers; and (vi) a memory coupled to the processor and comprising computer readable program code embodied in the memory that when executed by the at least one processor causes the at least one processor to perform operations comprising: (a) controlling/performing measurement via the sample analysis circuit of protein and/or peptide expression levels of a pre-defined set of gene products in said biological sample; (b) normalizing the gene product expression levels to generate normalized gene product expression values; retrieving from the storage circuit a fungal classifier, said classifier(s) comprising predefined weighting values for each of the pre-defined set of gene products; (c) entering the normalized gene product expression values into one or more fungal classifiers selected from the fungal classifier; (d) calculating a presence and/or etiology probability for the infection based upon said classifier(s), and optionally determining a threshold for the determination of fungal infection; and (e) controlling output via the input/output circuit of a determination of the presence of, or the etiology of, the fungal infection in the subject.

43. The system of embodiment 42, wherein the system comprises computer readable code to transform quantitative, or semi-quantitative, detection of gene product expression to a cumulative score or probability of the etiology of the infection.

44. The system of embodiment 42 or 43, wherein the system comprises an array platform, a gene product analyte hybridization or capture platform, multi-signal coded detector platform, a mass spectrometry platform, an amino acid sequencing platform, a nucleic acid sequencing platform, a PCR or other amplification platform, ELISA, Northern blot, SAGE, or a combination thereof.

45. The system of any one of embodiments 42-44, wherein the pre-defined set of gene products comprises from 2-146 gene products listed in Table 1.

46. The system of any one of embodiments 42-44, wherein the pre-defined set of gene products comprises from 2-187 gene products listed in Table 2.

47. The system of any one of embodiments 42-46, wherein the pre-defined set of analytes comprises from 2, 5, 8, 10, 15, 20, 25, 30, 30, 35, 40, 45, 50, 55, 60, 65, 70, 75, 80, 85, 90, 95, 100, 105, 110, 115, 120, 125, 130, 135, 140, 145 or 146 gene products (e.g., RNAs, proteins, and/or component peptides/epitopes) of the gene markers listed in Table 1 or 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 187 gene products of the gene markers listed in Table 2.

48. A method for determining the presence or absence of a fungal infection in a population of subjects comprising, consisting of, or consisting essentially of: (a) obtaining a biological sample from each of the subjects; (b) measuring on a platform expression levels of a pre-defined set of gene products in each of the biological samples; (c) normalizing the gene product expression levels to generate normalized expression values; (d) entering the normalized gene product expression values into one or more acute respiratory virus illness classifiers, said classifier(s) comprising pre-defined weighting values for each of the gene products of the pre-determined set of proteins and/or peptides for the platform, optionally wherein said classifier(s) are retrieved from one or more databases; and (e) calculating presence probability for a fungal infection based upon said normalized expression values and said classifier(s), to thereby determine the presence or absence of a fungal infection in the population of subjects.

49. A kit for determining the presence or absence of fungal etiology of an infection in a subject, or for detecting the presence or absence of a respiratory virus in a subject, comprising: (a) a means for extracting a biological sample; (b) a means for generating one or more arrays consisting of a plurality of antibodies or other analyte specific reagents for use in measuring gene product expression levels of a pre-defined set of gene products; and (c) optionally, instructions for use.

### EXAMPLES

### Example 1. Identification and Characterization of Gene Signatures: Approach and Methods

Invasive aspergillosis (IA) is a major cause of critical illness in immunocompromised (IC) patients. However, current fungal tests are limited. Disease-specific gene expression patterns in circulating host cells show promise as novel diagnostics, however it is unknown whether such a ‘signature’ exists for IA and the effect of iatrogenic immunosuppression on any such gene markers. The inventors of the methods provided in this disclosure conducted studies identifying host-based gene markers for diagnosis of Aspergillus as well as the effects of immunosuppression on such gene markers. The transcriptomic responses was examined in a murine model of inhalational Aspergillus fumigatus infection. Statistical and experimental approaches allowed the generation of a conserved signature of infection that functions with a high degree of accuracy across immunosuppressive states. These studies are described here. This work is also described in Steinbrink et al. (2020), “A transcriptional signature accurately identifies Aspergillus Infection across healthy and immunosuppressed states,” Translational Research 219, 1-12, which is incorporated herein by reference in its entirety for all purposes.

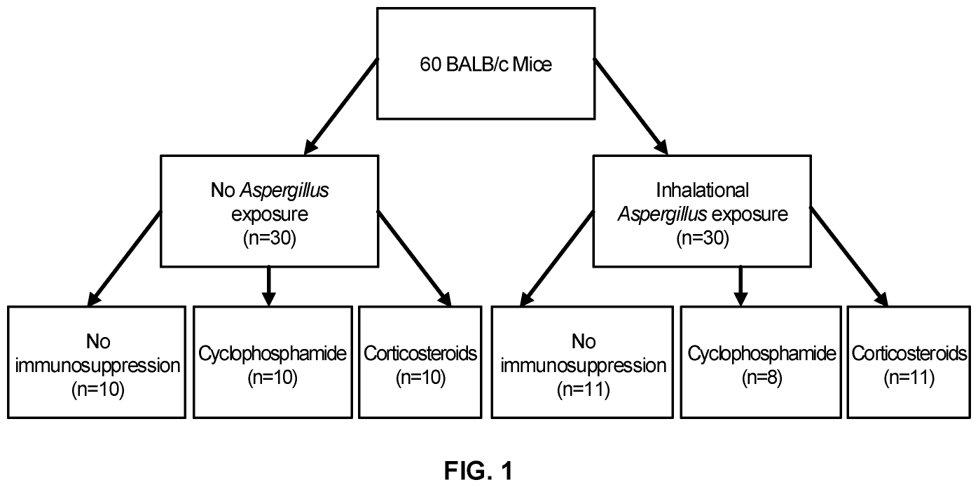

Experimental design: A total of 60 male BALB/c mice were separated into six experimental groups (FIG. 1). Mice were divided into groups based on immunosuppressed state: healthy mice, mice exposed to corticosteroids, and mice exposed to cyclophosphamide. Within each immunosuppression group, roughly half of the animals were exposed to Aspergillus fumigatus or placebo. Mice were sacrificed four days post-infection. All blood and tissue samples were collected on day +4. Samples were provided by National Institutes of Health under Contract No. N01-AI-30041. All protocols for murine work were approved by the institution's IACUC.

Immunosuppression and Aspergillus Exposure: Mice were immunosuppressed with intraperitoneal injection of cyclophosphamide (250 mg/kg on day −2 and 200 mg/kg on day +3 from infection) (n=18) or subcutaneous injection of cortisone acetate (250 mg/kg on day −2 and day +3 from infection) (n=21) using a standardized protocol for producing invasive aspergillosis (Sheppard et al. (2004), “Novel inhalational murine model of invasive pulmonary aspergillosis,” Antimicrobial agents and chemotherapy, 48(5):1908-11; Sheppard et al. (2006), “Standardization of an experimental murine model of invasive pulmonary aspergillosis,” Antimicrobial agents and chemotherapy, 50(10):3501-3). This immunosuppressive regimen produces leukopenia for up to 6 days, thus to prevent bacterial infection, mice received subcutaneous ceftazidime 50 mg/kg on day −2 from infection and continuing daily until the end of the study.

Mice in the Aspergillus infection group (n=30) were infected by 12 mL of 10⁹ conidia per mL AF293 aerosolized in an acrylic inhalation chamber for 1 hour on day 0. This model produces invasive pulmonary aspergillosis with histopathologic confirmation of tissue invasion in all three groups of mice (Sheppard et al. (2004), “Novel inhalational murine model of invasive pulmonary aspergillosis,” Antimicrobial agents and chemotherapy, 48(5):1908-11; Sheppard et al. (2006), “Standardization of an experimental murine model of invasive pulmonary aspergillosis,” Antimicrobial agents and chemotherapy, 50(10):3501-3). After sacrifice on day 4, lungs from each group were removed and homogenized in sterile saline. Determination of fungal burden in lung tissue was measured by colony-forming units (CFU) after plates were incubated at 37° C. for 24 hours.

RNA Preparation: Whole blood RNA isolation and β-globin reduction were carried out on 60 mice using the manufacturer's protocol (Mouse RiboPure and GLOBINclear, Ambion). The amount and purity of RNA yield was analyzed using a NanoDrop spectrophotometer (Thermo Fischer Scientific) and the integrity was analyzed using an Agilent Bioanalyzer. RNA from the 60 samples that met quality control checks (260/280 ratio >1.8, 260/230 ratio >1.0, and RNA integrity number >7) were used for microarray analysis. RNA was amplified and biotin-labeled using MessageAmp Premier RNA Amplification kit (Ambion) according to standard protocols at the Duke University Microarray Core facility. The Duke University Microarray Core performed amplification and hybridization onto Affymetrix murine 430A2.0 microarrays. Probe intensities were detected using Axon GenePix 4000B Scanner (Molecular Devices). Image files were generated using Affymetrix GeneChip Command Console software.

Differential Expression Analysis: Affymetrix microarray data was processed, underwent QC, and was normalized with the robust multi-array average method using the affy (Gautier et al., (2004), “affy—analysis of Affymetrix GeneChip data at the probe level,” Bioinformatics (Oxford, England), 20(3):307-15) Bioconductor (Gentleman et al. (2004), “Bioconductor: open software development for computational biology and bioinformatics. Genome biology,” 5(10):R80) package from the R statistical programming environment (available from www.r-project.org). Probes were filtered to exclude any probe not marked as present or marginal in at least one sample based on Affymetrix MAS 5.0 calls. Probes were annotated with the most current NetAffx Annotation from the Affymetrix website (www.affymetrix.com/support/technical/byproduct.affx?product=moe430A-20; Release 36, mm10). To assess for the presence of outliers and explore group structure, a principal components analysis was performed (data not shown here; see Supplementary Fig. S1 in Steinbrink et al., 2020) using the FactoMineR (Lê et al., (2008), “FactoMineR: An R Package for Multivariate Analysis,” 25(1):18) and factoextra (Mundt, factoextra: Extract and Visualize the Results of Multivariate Data Analyses. R package version 105. 2017) packages in R. Differential expression was carried out using a moderated t-statistic from the limma package (Ritchie et al. (2015), “limma powers differential expression analyses for RNA-sequencing and microarray studies,” Nucleic acids research, 43(7):e47). Two separate models were run to identify differentially expressed genes between infected and non-infected mice: 1) Control model, where only samples from non-immunocompromised animals were evaluated, and 2) Complete Model—entire data set with medication included as a cofactor included in the model. The false discovery rate was used to control for multiple hypothesis testing.

Pathway Analysis: Differentially-regulated pathways and gene ontology terms were identified using the Database for Annotation, Visualization, and Integrated Discovery (DAVID; Huang da et al. (2009), “Systematic and integrative analysis of large gene lists using DAVID bioinformatics resources,” Nature protocols, 4(1):44-57), Gene Set Enrichment Analysis (GSEA; Mootha et al. (2003), “PGC-1alpha-responsive genes involved in oxidative phosphorylation are coordinately downregulated in human diabetes,” Nature genetics, 34(3):267-73 and Ingenuity Pathway Analysis (IPA).

Aspergillus Infection Signature: An elastic net regularized logistic regression model was computed using all normalized probes post-filtering in the glmnet package in R (Friedman et al., (2010), “Regularization Paths for Generalized Linear Models via Coordinate Descent,” Journal of statistical software, 33(1):1-22). Alpha, which sets the balance between ridge and lasso regression, was set to 0.5. Leave-one-out or 10-fold cross-validation were used to determine the optimum lambda value that minimized the model error rate for Classifiers 1 and 2, respectively. Specifically, probe coefficients are based on the lambda value resulting in the minimum mean standard error for each model as determined by internal cross-validation (“lambda.min”). Additionally, two separate elastic net models were developed. The first model (Control, Classifier 1) included only samples without immunosuppressing medication. The second (Complete, Classifier 2) included the filtered data set after controlling for immunosuppressing medication by supplanting the normalized gene expression values of each gene with the global mean for that gene plus the residuals derived from a linear regression of immunosuppressive therapy on expression. Model performance and stability was evaluated with 10-fold cross-validation (10-fold CV) for the Complete model and leave-one-out cross-validation (LOO CV) for the Control analysis. This resulted in 10 separate models (10-fold CV) or n-separate models (LOO CV), due to smaller sample size. A full model using the entire data set was computed for all models. Model coefficients from the full Control model were projected onto the Complete (unadjusted) data set to assess model performance.

Comparison with Invasive Candidiasis: We next compared the host response to IA to the response to Candida infection using transcriptomic data from peripheral blood samples taken from a murine model of invasive candidiasis (Zaas et al. (2010), “Blood gene expression signatures predict invasive candidiasis,” Science translational medicine, 2(21):21ra17). Differential expression analysis was carried out for all probes using a moderated t-statistic from the limma package (Ritchie et al. (2015), “limma powers differential expression analyses for RNA-sequencing and microarray studies,” Nucleic acids research, 43(7):e47). The false discovery rate (FDR) was used to control for multiple hypothesis testing. Next, a general linear model was also run on the Candida data using only those probes included in the Aspergillus Control Classifier (Classifier 1). As a means of assessing overlap in biological functions between the Candida and Aspergillus results, IPA (Qiagen Bioinformatics) was run on the intersection of probes from the limma differential expression analysis that were significant at an FDR of 0.01 or smaller.

### Example 2. Clinical Outcomes

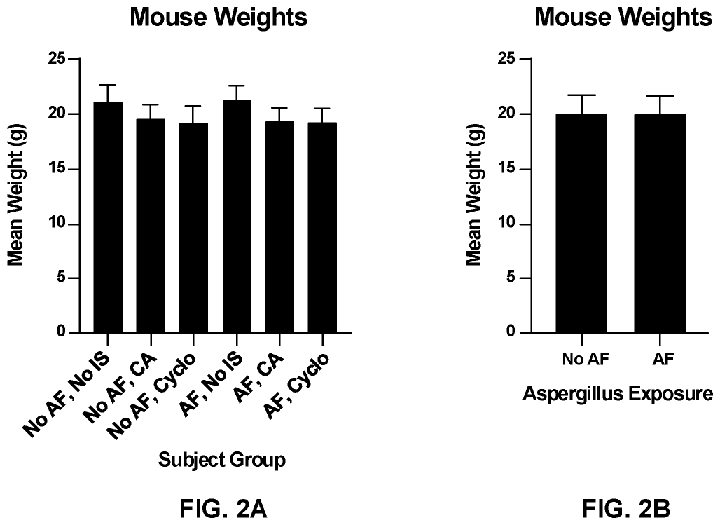

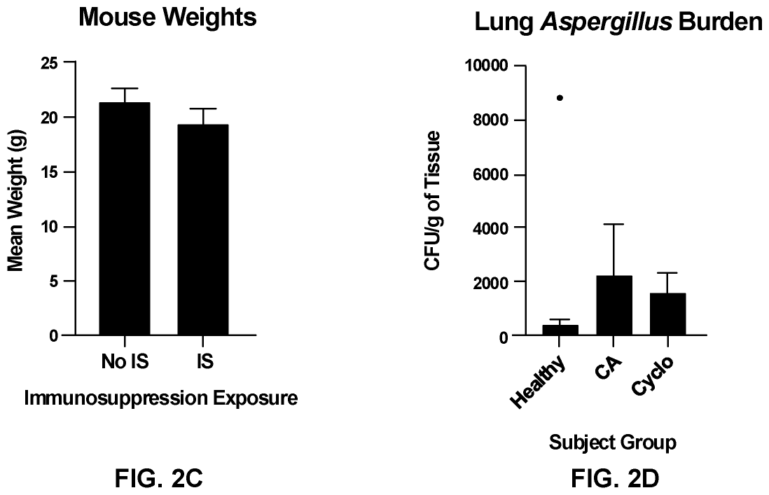

Mice within each of three immunosuppression groups (healthy, corticosteroid, cyclophosphamide) were exposed to inhalational Aspergillus or placebo and monitored for 4 days. At the end of the 4 days, mice were sacrificed and blood and tissue samples were obtained. The average weight of mice exposed to Aspergillus (irrespective of immunosuppression) was slightly lower in the group not exposed to Aspergillus, but this was not significant (p=0.86, Student's t test; FIG. 2B). The average weight of mice that received immunosuppressive medication was less than those who received no immunosuppression, irrespective of Aspergillus exposure, and this was significant (p<0.0001, Student's t test; FIG. 2C). Weights did not differ significantly based on the type of immunosuppression (FIG. 2A).

Fungal burden in the lungs was measured for the three cohorts of infected mice at the time of sacrifice 4 days after inhalation by CFU. Lung fungal burden was higher in mice that were immunosuppressed, although this did not reach significance (p=0.29, Student's t test; FIG. 2D). There was one outlier in the non-immunosuppressed mice with a lung fungal burden of 8710 CFU/g. The remaining values had a mean of 407.27 (range 143-899) CFU/g. Of the infected mice that received corticosteroids, the mean fungal tissue burden was 2254.55 (range 525-6540) CFU/g. Of the infected mice that received cyclophosphamide, the mean was 1626 (range 469-2780) CFU/g.

### Example 3. Gene Expression Signature Discriminating Between Mice with IA and Uninfected Mice in the Absence of Immunosuppression

To characterize the host transcriptional response to IA, a linear model with a moderated test statistic was utilized to assess the differential expression of genes between infected (n=11) and uninfected (n=10) mice. This identified 2,826 probe sets reflecting 2,718 genes that were differentially expressed (adjusted p<0.05) when examining Aspergillus-infected compared to uninfected animals. Of these, 1,672 genes were up-regulated. The most significantly up-regulated genes clustered into the following immune-related biological pathways (via Gene Ontology, GO): apoptotic process, negative regulation of apoptotic process, immune system process, inflammatory response, adaptive immune response, positive regulation of NF-kappa-B signaling, response to oxidative stress, cellular response to cytokine stimulus, cellular response to interleukin-4, T cell receptor signaling pathway, T cell activation, T cell differentiation, activated T cell proliferation, and positive regulation of macrophage chemotaxis.

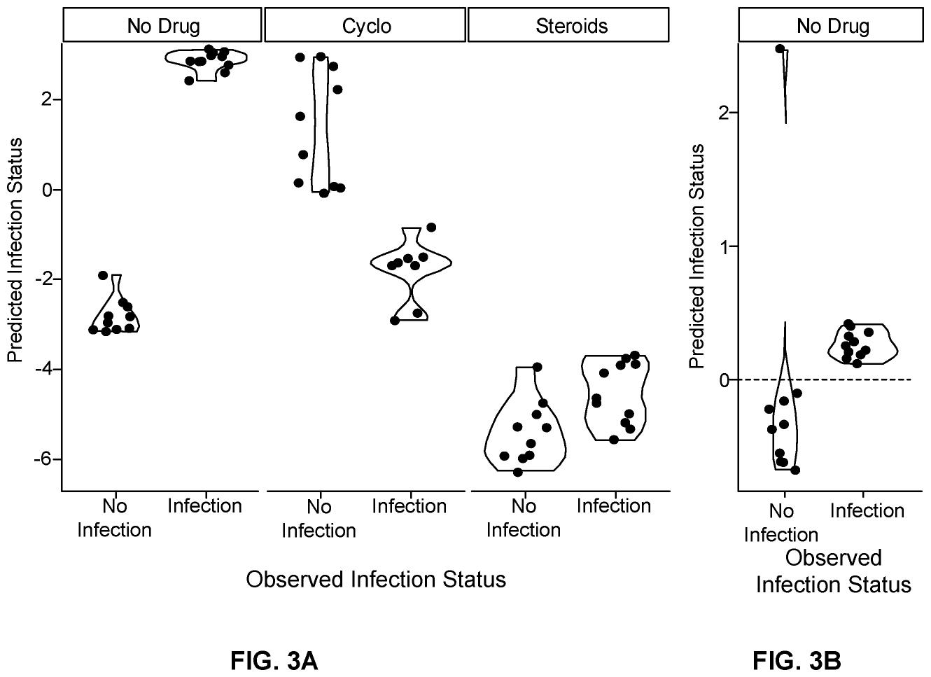

To identify a transcriptomic signature associated with A. fumigatus infection, an elastic net regularized logistic regression analysis was performed to determine a set of genes that distinguished infected from uninfected samples. A transcriptomic classifier of 152 probes representing 146 genes was generated which was able to differentiate mice with IA from healthy mice with an AUC of 1 in the absence of immunosuppression (hereafter referred to as Classifier 1, listed in Table 1). See FIGS. 3A and 3B.

TABLE 1
Genes included in Classifier 1.

| | | | |
|---|---|---|---|
| Probe ID | Gene Symbol | Gene Name | logFC |

Upregulated Genes

| | | | |
|---|---|---|---|
| 1416809_at | Cyp3a11 | cytochrome P450, family 3, subfamily a, | 1.55129676 |
| | | polypeptide 11 | |
| 1455972_x_at | Hadh | hydroxyacyl-Coenzyme A dehydrogenase | 0.69505514 |
| 1449394_at | Slco1b2 | solute carrier organic anion transporter | 0.58684191 |
| | | family, member 1b2 | |
| 1455988_a_at | Cct6a | chaperonin containing Tcp1, subunit 6a (zeta) | 0.50909821 |
| 1452388_at | Hspa1a | heat shock protein 1A | 0.4324812 |
| 1417766_at | Cyb5b | cytochrome b5 type B | 0.41754077 |
| 1455855_x_at | Hnrnpab | heterogeneous nuclear ribonucleoprotein A/B | 0.40428288 |
| 1433508_at | Klf6 | Kruppel-like factor 6 | 0.36878305 |
| 1452213_at | Tex2 | testis expressed gene 2 | 0.36479111 |
| 1451208_at | Etf1 | eukaryotic translation termination factor 1 | 0.34996413 |
| 1420772_a_at | Tsc22d3 | TSC22 domain family, member 3 | 0.34479577 |
| 1460331_at | Tm9sf2 | transmembrane 9 superfamily member 2 | 0.34459799 |
| 1424424_at | Slc39a1 | solute carrier family 39 (zinc transporter), | 0.32077174 |
| | | member 1 | |
| 1415831_at | Psmd2 | proteasome (prosome, macropain) 26S subunit, | 0.31255885 |
| | | non-ATPase, 2 | |
| 1427032_at | Herc4 | hect domain and RLD 4 | 0.30731907 |
| 1423517_at | Cct6a | chaperonin containing Tcp1, subunit 6a (zeta) | 0.29851498 |
| 1450963_at | Hnrnpf | heterogeneous nuclear ribonucleoprotein F | 0.28954877 |
| 1448710_at | Cxcr4 | chemokine (C-X-C motif) receptor 4 | 0.28218569 |
| 1456040_at | Sf3b2 | splicing factor 3b, subunit 2 | 0.27348735 |
| 1437192_x_at | Vdac1 | voltage-dependent anion channel 1 | 0.25568748 |
| 1455788_x_at | Poldip3 | polymerase (DNA-directed), delta interacting | 0.22509694 |
| | | protein 3 | |
| 1429514_at | Plpp3 | phospholipid phosphatase 3 | 0.2135838 |
| 1423545_a_at | Zfp207 | zinc finger protein 207 | 0.20810918 |
| 1448107_x_at | Klk1 | kallikrein 1 | 0.20167999 |
| 1437099_x_at | Hnrnpf | heterogeneous nuclear ribonucleoprotein F | 0.19578093 |
| 1417225_at | Arl6ip5 | ADP-ribosylation factor-like 6 | 0.18722003 |
| | | interacting protein 5 | |
| 1423667_at | Mat2a | methionine adenosyltransferase II, alpha | 0.17642225 |
| 1435086_s_at | Klhdc2 | kelch domain containing 2 | 0.17176944 |
| 1448673_at | Pvrl3 | poliovirus receptor-related 3 | 0.16751959 |
| 1449360_at | Csf2rb2 | colony stimulating factor 2 receptor, | 0.1673807 |
| | | beta 2, low-affinity (granulocyte-macrophage) | |
| 1426682_at | Cnot6 | CCR4-NOT transcription complex, | 0.16710883 |
| | | subunit 6 | |
| 1417398_at | Rras2 | related RAS viral (r-ras) oncogene | 0.16072672 |
| | | homolog 2 | |
| 1452182_at | Galnt2 | UDP-N-acetyl-alpha-D-galactosamine:polypeptide | 0.15351468 |
| | | N-acetylgalactosaminyltransferase 2 | |
| 1422576_at | Atxn10 | ataxin 10 | 0.15204377 |
| 1426343_at | Stt3b | STT3, subunit of the oligosaccharyltransferase | 0.13994088 |
| | | complex, homolog B (S. cerevisiae) | |
| 1426342_at | Stt3b | STT3, subunit of the oligosaccharyltransferase | 0.13842874 |
| | | complex, homolog B (S. cerevisiae) | |
| 1416046_a_at | Fuca2 | fucosidase, alpha-L- 2, plasma | 0.12784693 |
| 1449846_at | Ear12///Ear2///Ear3 | eosinophil-associated, ribonuclease A family, | 0.12449441 |
| | | member 12///eosinophil-associated, ribonuclease | |
| | | A family, member 2///eosinophil-associated, | |
| | | ribonuclease A family, member 3 | |
| 1460460_a_at | Gorasp2 | golgi reassembly stacking protein 2 | 0.12356174 |
| 1427898_at | Rnf6 | ring finger protein (C3H2C3 type) 6 | 0.11868658 |
| 1424039_at | Saraf | store-operated calcium entry-associated | 0.11576948 |
| | | regulatory factor | |
| 1424980_s_at | Aph1a | anterior pharynx defective 1a homolog | 0.11099402 |
| | | (C. elegans) | |
| 1452152_at | Clint1 | clathrin interactor 1 | 0.1028463 |
| 1448580_at | Glg1 | golgi apparatus protein 1 | 0.09384171 |
| 1456006_at | Bcl2l11 | BCL2-like 11 (apoptosis facilitator) | 0.09371802 |
| 1423687_a_at | Man2c1 | mannosidase, alpha, class 2C, member 1 | 0.08629966 |
| 1419092_a_at | Slk | STE20-like kinase | 0.08297865 |
| 1421731_a_at | Fen1 | flap structure specific endonuclease 1 | 0.08199737 |
| 1416106_at | Kti12 | KTI12 homolog, chromatin associated | 0.08079984 |
| | | (S. cerevisiae) | |
| 1427906_at | 1110037F02Rik | RIKEN cDNA 1110037F02 gene | 0.0788807 |
| 1423817_s_at | Use1 | unconventional SNARE in the ER 1 | 0.07297964 |
| | | homolog (S. cerevisiae) | |
| 1416087_at | Ap1s1 | adaptor protein complex AP-1, sigma 1 | 0.06771846 |
| 1422660_at | Gm15453///Rbm3 | predicted gene 15453///RNA binding | 0.06530285 |
| | | motif protein 3 | |
| 1426870_at | Fbxo33 | F-box protein 33 | 0.06526147 |
| 1437525_a_at | Polr3a | polymerase (RNA) III (DNA directed) | 0.06449351 |
| | | polypeptide A | |
| 1426871_at | Fbxo33 | F-box protein 33 | 0.0552003 |
| 1417628_at | Supt6 | suppressor of Ty 6 | 0.05066466 |
| 1418840_at | Pdcd4 | programmed cell death 4 | 0.04939937 |
| 1433942_at | Myo6 | myosin VI | 0.03915114 |
| 1421450_a_at | Map3k4 | mitogen-activated protein kinase | 0.03833506 |
| | | kinase kinase 4 | |
| 1451544_at | Tapbpl | TAP binding protein-like | 0.03341531 |
| 1418455_at | Copz2 | coatomer protein complex, subunit zeta 2 | 0.02563244 |
| 1452703_at | Ahcyl2 | S-adenosylhomocysteine hydrolase-like 2 | 0.01795472 |
| 1451413_at | Cast | calpastatin | 0.01749003 |
| 1427385_s_at | Actn1 | actinin, alpha 1 | 0.01679797 |
| 1419128_at | Itgax | integrin alpha X | 0.01102072 |
| 1439771_s_at | D13Ertd608e | DNA segment, Chr 13, ERATO Doi 608, expressed | 0.0110196 |
| 1418658_at | Rmdn1 | regulator of microtubule dynamics 1 | 0.00822705 |
| 1416505_at | Nr4a1 | nuclear receptor subfamily 4, group | 0.00151475 |
| | | A, member 1 | |
| 1435357_at | Rsrp1 | arginine/serine rich protein 1 | 0.00117443 |

Downregulated Genes

| | | | |
|---|---|---|---|
| 1422455_s_at | Nsf | N-ethylmaleimide sensitive fusion | −0.0069127 |
| | | protein | |
| 1452697_at | Ctdp1 | CTD (carboxy-terminal domain, RNA | −0.0086058 |
| | | polymerase II, polypeptide A) | |
| | | phosphatase, subunit 1 | |
| 1448731_at | Il10ra | interleukin 10 receptor, alpha | −0.0096126 |
| 1416188_at | Gm2a | GM2 ganglioside activator protein | −0.0103885 |
| 1422966_a_at | Tfrc | transferrin receptor | −0.0254664 |
| 1449296_a_at | Cnp | 2′,3′-cyclic nucleotide | −0.0280805 |
| | | 3′ phosphodiesterase | |
| 1423827_s_at | Noc41 | nucleolar complex associated 4 | −0.0343738 |
| | | homolog (S. cerevisiae) | |
| 1450757_at | Cdh11 | cadherin 11 | −0.0355886 |
| 1449254_at | Spp1 | secreted phosphoprotein 1 | −0.0409736 |
| 1427412_s_at | Rapgef6 | Rap guanine nucleotide exchange | −0.0421985 |
| | | factor (GEF) 6 | |
| 1460200_s_at | Lztfl1 | leucine zipper transcription | −0.0424654 |
| | | factor-like 1 | |
| 1421402_at | Mta3 | metastasis associated 3 | −0.0524082 |
| 1427439_s_at | Prmt5 | protein arginine N-methyltransferase 5 | −0.0527715 |
| 1427381_at | Irg1 | immunoresponsive gene 1 | −0.0566286 |
| 1451206_s_at | Cytip | cytohesin 1 interacting protein | −0.0576289 |
| 1421299_a_at | Lef1 | lymphoid enhancer binding factor 1 | −0.0704712 |
| 1420817_at | Ywhag | tyrosine 3-monooxygenase/tryptophan | −0.0748744 |
| | | 5-monooxygenase activation protein, | |
| | | gamma polypeptide | |
| 1451043_at | Nek6 | NIMA (never in mitosis gene a)-related | −0.0761472 |
| | | expressed kinase 6 | |
| 1417371_at | Peli1 | pellino 1 | −0.1051182 |
| 1423608_at | Itm2a | integral membrane protein 2A | −0.1160327 |
| 1429921_at | 9530068E07Rik | RIKEN cDNA 9530068E07 gene | −0.1284912 |
| 1422391_at | Vmn1r11 | vomeronasal 1 receptor 11 | −0.1425121 |
| 1448199_at | Ankrd10 | ankyrin repeat domain 10 | −0.1447443 |
| 1460338_a_at | Crlf3 | cytokine receptor-like factor 3 | −0.1463586 |
| 1448361_at | Ttc3 | tetratricopeptide repeat domain 3 | −0.1475267 |
| 1427335_at | Tmem260 | transmembrane protein 260 | −0.1590426 |
| 1417589_at | Galnt3 | UDP-N-acetyl-alpha-D-galactosamine:polypeptide | −0.1607985 |
| | | N-acetylgalactosaminyltransferase 3 | |
| 1422967_a_at | Tfrc | transferrin receptor | −0.1757556 |
| 1455214_at | Mitf | microphthalmia-associated | −0.1909813 |
| | | transcription factor | |
| 1417701_at | Ppp1r14c | protein phosphatase 1, regulatory | −0.1936346 |
| | | (inhibitor) subunit 14c | |
| 1425660_at | Btbd3 | BTB (POZ) domain containing 3 | −0.1964939 |
| 1417753_at | Pkd2 | polycystic kidney disease 2 | −0.1975538 |
| 1427168_a_at | Col14a1 | collagen, type XIV, alpha 1 | −0.2098561 |
| 1432155_at | Wasl | Wiskott-Aldrich syndrome-like (human) | −0.2100399 |
| 1425785_a_at | Txk | TXK tyrosine kinase | −0.2174612 |
| 1423126_at | Atp1b3 | ATPase, Na+/K+ transporting, | −0.2214641 |
| | | beta 3 polypeptide | |
| 1453985_at | Ercc612 | excision repair cross-complementing rodent | −0.2359139 |
| | | repair deficiency, complementation | |
| | | group 6 like 2 | |
| 1431213_a_at | Gm3579///Gm40514///Gm40814/// | predicted gene 3579///predicted gene, | −0.2370748 |
| | Gm40991///Gm42035///Gm42102/// | 40514///predicted gene, 40814///predicted gene, | |
| | LOC105244034 | 40991///predicted gene, 42035///predicted gene, | |
| | | 42102///uncharacterized LOCI05244034 | |
| 1449212_at | Pip | prolactin induced protein | −0.23914 |
| 1420759_s_at | Zfy1///Zfy2 | zinc finger protein 1, Y-linked///zinc finger | −0.2415667 |
| | | protein 2, Y-linked | |
| 1450331_s_at | Vmn2r34///Vmn2r42///Vmn2r45 | vomeronasal 2, receptor 34///vomeronasal 2, | −0.2428143 |
| | | receptor 42///vomeronasal 2, receptor 45 | |
| 1438220_at | Foxj3 | forkhead box J3 | −0.2483283 |
| 1422175_at | Mmpla | matrix metallopeptidase 1a | −0.2543012 |
| | | (interstitial collagenase) | |
| 1420873_at | Gm4887///Twfl | predicted gene 4887///twinfilin, actin- | −0.2692545 |
| | | binding protein, homolog 1 (Drosophila) | |
| 1418163_at | Tlr4 | toll-like receptor 4 | −0.2712715 |
| 1435375_at | Fam105a | family with sequence similarity | −0.2737653 |
| | | 105, member A | |
| 1422345_s_at | Mageb1///Mageb2///Mageb3 | melanoma antigen, family B, 1///melanoma | −0.2827112 |
| | | antigen, family B, 2///melanoma antigen, | |
| | | family B, 3 | |
| 1447977_x_at | 0610010B08Rik///Gm11007/// | RIKEN cDNA 0610010B08 gene///predicted gene | −0.2834026 |
| | Gm14308///Gm14430/// | 11007///predicted gene 14308///predicted gene | |
| | Gm14434///Gm4724 | 14430///predicted gene 14434///predicted | |
| | | gene 4724 | |
| 1435628_x_at | BC005512 | cDNA sequence BC005512 | −0.2852137 |
| 1439045_x_at | Tc2n | tandem C2 domains, nuclear | −0.2926997 |
| 1421222_at | Fip1l1 | FIP1 like 1 (S. cerevisiae) | −0.2999038 |
| 1460416_s_at | Csprs///Gm15433///Gm2666/// | component of Sp100-rs///predicted pseudogene | −0.3073112 |
| | Gm7609///LOC100041903/// | 15433///predicted gene 2666///predicted | |
| | LOC100503923///LOC101055758 | pseudogene 7609///proteinase-activated | |
| | | receptor 1-like///proteinase-activated | |
| | | receptor 1-like///component of Sp100-rs | |
| 1426322_a_at | Kcnmb2 | potassium large conductance calcium-activated | −0.3079439 |
| | | channel, subfamily M, beta member 2 | |
| 1460292_a_at | Smarca1 | SWI/SNF related, matrix associated, actin | −0.3122081 |
| | | dependent regulator of chromatin, | |
| | | subfamily a, member 1 | |
| 1426570_a_at | Frk | fyn-related kinase | −0.3132723 |
| 1417262_at | Ptgs2 | prostaglandin-endoperoxide synthase 2 | −0.3210158 |
| 1433758_at | Nisch | nischarin | −0.3254956 |
| 1438748_at | Cacul1 | CDK2 associated, cullin domain 1 | −0.3326497 |
| 1422939_at | Serpinb3b///Serpinb3c | serine (or cysteine) peptidase inhibitor, | −0.3412673 |
| | | clade B (ovalbumin), member 3B///serine | |
| | | (or cysteine) peptidase inhibitor, clade | |
| | | B, member 3C | |
| 1436222_at | Gas5 | growth arrest specific 5 | −0.3451841 |
| 1452899_at | Rian | RNA imprinted and accumulated in nucleus | −0.3529845 |
| 1421979_at | Phex | phosphate regulating endopeptidase | −0.353347 |
| | | homolog, X-linked | |
| 1425813_at | Pign | phosphatidylinositol glycan anchor | −0.359102 |
| | | biosynthesis, class N | |
| 1450293_at | Mageb3 | melanoma antigen, family B, 3 | −0.3753081 |
| 1422961_at | Nat3 | N-acetyltransferase 3 | −0.3787963 |
| 1450962_at | Pdha2 | pyruvate dehydrogenase E1 alpha 2 | −0.3839711 |
| 1454686_at | 6430706D22Rik/// | RIKEN cDNA 6430706D22 gene///RIKEN | −0.3925624 |
| | A730008H23Rik///Hjurp | cDNA A730008H23 gene///Holliday | |
| | | junction recognition protein | |
| 1424608_a_at | Bzw2 | basic leucine zipper and W2 domains 2 | −0.4065812 |
| 1422940_x_at | Serpinb3b///Serpinb3c | serine (or cysteine) peptidase inhibitor, | −0.4263653 |
| | | clade B (ovalbumin), member 3B///serine | |
| | | (or cysteine) peptidase inhibitor, clade | |
| | | B, member 3C | |
| 1425083_at | Otor | otoraplin | −0.4304466 |
| 1422617_at | Gm10058///Gm10096///Gm10147/// | predicted gene 10058///predicted gene | −0.4325473 |
| | Gm10230///Gm10486///Gm10488/// | 10096///predicted gene 10147///predicted | |
| | Gm14525///Gm14632///Gm14819/// | gene 10230///predicted gene 10486/// | |
| | Gm2012///Gm2030///Gm4297/// | predicted gene 10488///predicted gene | |
| | Gm5169///Gm5934 ///Gm6121 | 14525///predicted gene 14632///predicted | |
| | | gene 14819///predicted gene 2012/// | |
| | | predicted gene 2030///predicted gene | |
| | | 4297///predicted gene 5169///predicted | |
| | | gene 5934///predicted gene 6121 | |
| 1422618_x_at | Gm10058///Gm10096///Gm10147/// | predicted gene 10058///predicted gene | −0.4629588 |
| | Gm10230///Gm10486///Gm10488/// | 10096///predicted gene 10147///predicted | |
| | Gm14525///Gm14632///Gm14819/// | gene 10230///predicted gene 10486/// | |
| | Gm2012///Gm2030///Gm4297/// | predicted gene 10488///predicted gene | |
| | Gm5169///Gm5934 ///Gm6121 | 14525///predicted gene 14632///predicted | |
| | | gene 14819///predicted gene 2012/// | |
| | | predicted gene 2030///predicted gene | |
| | | 4297///predicted gene5169///predicted | |
| | | gene 5934///predicted gene 6121 | |
| 1448821_at | Tyr | tyrosinase | −0.5015319 |
| 1441147_at | D3Ertd229e | DNA segment, Chr 3, ERATO Doi 229, expressed | −0.50903 |
| 1449832_at | 1700091H14Rik | RIKEN cDNA 1700091H14 gene | −0.5899165 |
| 1424537_at | Gm10921///Gm10922///Gm14345/// | predicted gene 10921///predicted gene | −0.595637 |
| | Gm14346///Gm14347///Gm14351/// | 10922///predicted gene 14345///predicted | |
| | Gm14367///Gm14374///Gm21608/// | gene 14346///predicted gene 14347/// | |
| | Gm21645///Gm21681///Gm21699/// | predicted gene 14351///predicted gene | |
| | Gm21950///Gm21951///Gm2777/// | 14367///predicted gene 14374///predicted | |
| | Gm2784///Gm2799///Gm2825/// | gene, 21608///predicted gene, 21645/// | |
| | Gm2863///Gm2913///Gm2927/// | predicted gene, 21681///predicted gene, | |
| | Gm2964///Gm3701///Gm3706/// | 21699///predicted gene, 21950///predicted | |
| | Gm3750///Gm3763///Gm5925/// | gene, 21951///predicted gene 2777/// | |
| | Gmcl11///LOC100048813 | predicted gene 2784///predicted gene | |
| | | 2799///predicted gene 2825///predicted | |
| | | gene 2863///predicted gene 2913///predicted | |
| | | gene 2927///predicted gene 2964///predicted | |
| | | gene 3701///predicted gene 3706///predicted | |
| | | gene 3750///predicted gene 3763///germ cell- | |
| | | less homolog 1 family pseudogene///germ cell- | |
| | | less homolog 1 (Drosophila)-like/// | |
| | | germ cell-less-like | |

### Example 4. The Presence of Immunosuppression Confounds the Gene Expression Signature for A. fumigatus

In the presence of immunosuppression there were marked changes in the nature of the host transcriptional response to IA. 1,391 of 16,468 overall genes (8.45%) in mice infected with Aspergillus were upregulated by at least 20% when comparing mean expression in cyclophosphamide exposure to those with no immunosuppression, with a maximum fold change of 3.78. 372 genes (2.26%) were downregulated, with a minimum fold change of 0.46. In the presence of corticosteroids, overall gene expression fold change in infected mice ranged from 2.50 to 0.45 compared to those without immunosuppression, with 418 genes (2.54%) upregulated and 302 genes (1.83%) downregulated.

In order to assess the performance of this transcriptional signature in the presence of immunosuppression, the transcriptional signature derived from non-immunosuppressed mice (Classifier 1) was projected onto the combined dataset of all groups (no immunosuppression; cyclophosphamide; corticosteroids). As can be seen in FIG. 3A, while this model accurately discriminates between all infected and uninfected animals in the absence of immunocompromising medication, when applied to mice that did receive immunosuppression, this model failed to accurately discriminate between the presence or absence of infection, regardless of the type of immunosuppression. While the model could distinguish that there were two distinct groups in the presence of cyclophosphamide, it could not accurately identify which samples were infected. The model could not clearly distinguish between the two groups in the presence of corticosteroids.

### Example 5. Controlling for Immunosuppressive Effects Permits Derivation of a Conserved Gene Expression Signature for A. fumigatus Infection

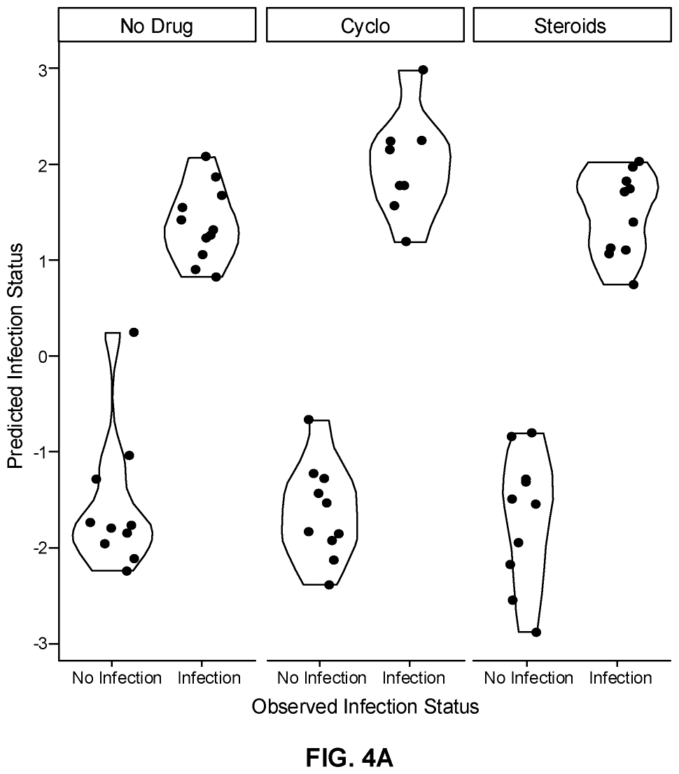

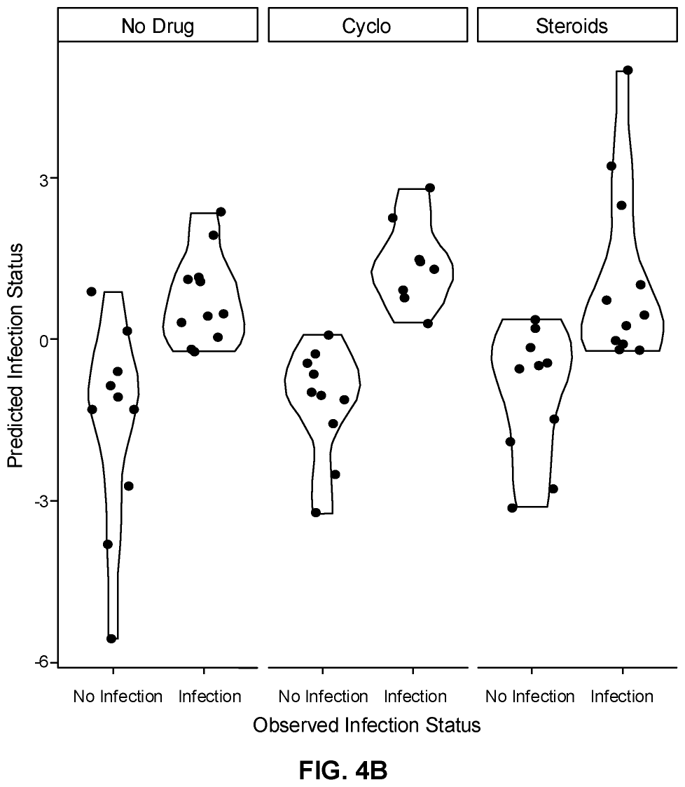

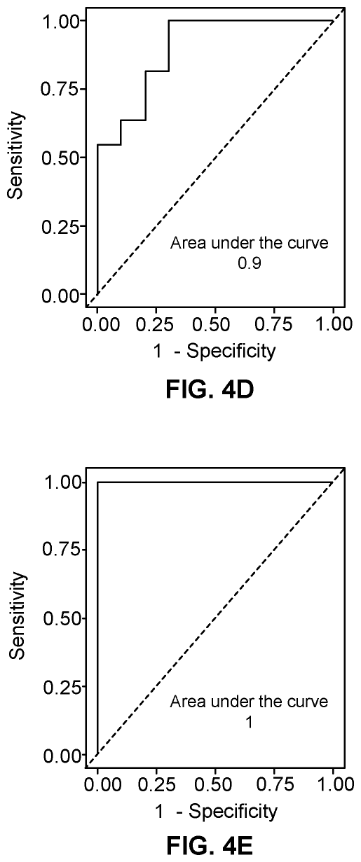

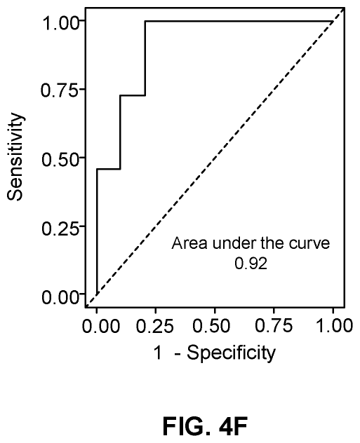

The data demonstrate that the presence of cyclophosphamide or corticosteroids altered the expression of genes driving some core components of the host response to Aspergillus infection, which directly affected performance of the initial classifier. Controlling for immunosuppressing medication was performed by replacing normalized gene expression values with the mean plus residuals derived from a linear model of medication on gene expression. This resulted in a transcriptomic classifier of 199 probe sets representing 187 genes (Classifier 2, listed in Table 2). As demonstrated in FIG. 4A, when controlling for medication effect, the resulting Classifier 2 distinguished between infected and uninfected states in both immunosuppressed conditions as well as in non-immunosuppressed animals. A heatmap was created showing the z-score transformed normalized expression value of genes included in the full model (heatmap not shown here; see FIG. 4C in Steinbrink et al., 2020). Model performance was evaluated using 10-fold cross-validation (FIG. 4B) which yielded an AUC of 0.92 for no medication, 1 for cyclophosphamide, and 0.9 for steroids (FIGS. 4D, 4E, and 4F).

There were 18 genes that overlapped between Classifier 1 and 2, and thus were components of the host response to IA that seemed to be minimally affected by immunocompromise. These were not clustered into any significant immune pathways. However, they did include some genes reflective of host immune and inflammatory functions—including SerpinB3, which has been demonstrated to offer protection from oxidative injury due to particular chemotherapeutic drugs (Ciscato et al. (2014), “SERPINB3 protects from oxidative damage by chemotherapeutics through inhibition of mitochondrial respiratory complex I,” Oncotarget, 5(9):2418-27; Vidalino et al. (2009); “SERPINB3, apoptosis and autoimmunity,” Autoimmunity reviews, 9(2):108-12), MMP1, which is involved in the inflammatory response and cytokine modulation (Van Lint and Libert (2007), “Chemokine and cytokine processing by matrix metalloproteinases and its effect on leukocyte migration and inflammation,” Journal of leukocyte biology, 82(6):1375-81; Tasaki et al. (2003), “Pro-inflammatory cytokine-induced matrix metalloproteinase-1 (MMP-1) secretion in human pancreatic periacinar myofibroblasts,” Pancreatology: official journal of the International Association of Pancreatology (TAP), 3(5):414-21), PIP, demonstrated to have an impact on Thl cell-mediated immunity (Li et al. (2015), “Deficiency of prolactin-inducible protein leads to impaired Thl immune response and susceptibility to Leishmania major in mice,” European journal of immunology, 45(4):1082-91), and MITF, which suppresses generalized response to inflammation (Riesenberg et al. (2015), “MITF and c-Jun antagonism interconnects melanoma dedifferentiation with pro-inflammatory cytokine responsiveness and myeloid cell recruitment,” Nature communications, 6:8755).

TABLE 2
Genes included in Classifier 2.

| | | | |
|---|---|---|---|
| Probe ID | Gene Symbol | Gene Name | logFC |

Upregulated Genes

| | | | |
|---|---|---|---|
| 1418858_at | Aox3 | aldehyde oxidase 3 | 1.38872148 |
| 1456733_x_at | Serpinh1 | serine (or cysteine) peptidase inhibitor, clade H, member 1 | 0.57832789 |
| 1419918_at | Tmed7 | transmembrane emp24 protein transport domain containing 7 | 0.47511689 |
| 1449232_at | Gata1 | GATA binding protein 1 | 0.45735395 |
| 1416958_at | Nr1d2 | nuclear receptor subfamily 1, group D, member 2 | 0.4439457 |
| 1420819_at | Sla | src-like adaptor | 0.43612244 |
| 1452388_at | Hspa1a | heat shock protein 1A | 0.4324812 |
| 1428074_at | Tmeml58 | transmembrane protein 158 | 0.42419927 |
| 1452249_at | Prickle1 | prickle homolog 1 (Drosophila) | 0.40564791 |
| 1455855_x_at | Hnrnpab | heterogeneous nuclear ribonucleoprotein A/B | 0.40428288 |
| 1451867_x_at | Arhgap6 | Rho GTPase activating protein 6 | 0.39531997 |
| 1423685_at | Aars | alanyl-tRNA synthetase | 0.38510731 |
| 1456036_x_at | Gsto1 | glutathione S-transferase omega 1 | 0.37216439 |
| 1425364_a_at | Slc3a2 | solute carrier family 3 (activators of dibasic and neutral amino | 0.36962641 |
| | | acid transport), member 2 | |
| 1455073_at | Cdadc1 | cytidine and dCMP deaminase domain containing 1 | 0.36709992 |
| 1416262_at | Tmem19 | transmembrane protein 19 | 0.36393943 |
| 1434884_at | Mtdh | metadherin | 0.36262839 |
| 1435659_a_at | Tpi1 | triosephosphate isomerase 1 | 0.3603068 |
| 1449851_at | Per1 | period circadian clock 1 | 0.34828324 |
| 1438557_x_at | Dnpep | aspartyl aminopeptidase | 0.33667463 |
| 1433563_s_at | Derl1 | Der1-like domain family, member 1 | 0.33061829 |
| 1452059_at | Slc35f5 | solute carrier family 35, member F5 | 0.32862119 |
| 1422826_at | Igfals | insulin-like growth factor binding protein, acid labile subunit | 0.32615762 |
| 1424424_at | Slc39a1 | solute carrier family 39 (zinc transporter), member 1 | 0.32077174 |
| 1452709_at | Poldip3 | polymerase (DNA-directed), delta interacting protein 3 | 0.3176235 |
| 1426810_at | Kdm3a | lysine (K)-specific demethylase 3A | 0.31308049 |
| 1420012_at | Xbp1 | X-box binding protein 1 | 0.30915814 |
| 1424182_at | Acat1 | acetyl-Coenzyme A acetyltransferase 1 | 0.29422739 |
| 1422478_a_at | Acss2 | acyl-CoA synthetase short-chain family member 2 | 0.28684903 |
| 1437723_s_at | Derl1 | Der1-like domain family, member 1 | 0.28595572 |
| 1423386_at | Psmd9 | proteasome (prosome, macropain) 26S subunit, non-ATPase, 9 | 0.27242939 |
| 1420037_at | Atp5a1 | ATP synthase, H+ transporting, mitochondrial F1 complex, alpha | 0.26794327 |
| | | subunit 1 | |
| 1419256_at | Sptbn1 | spectrin beta, non-erythrocytic 1 | 0.26522107 |
| 1437192_x_at | Vdac1 | voltage-dependent anion channel 1 | 0.25568748 |
| 1451624_a_at | Phospho2 | phosphatase, orphan 2 | 0.25401713 |
| 1437837_x_at | Poldip3 | polymerase (DNA-directed), delta interacting protein 3 | 0.24897594 |
| 1456193_x_at | Gm7338///Gpx4 | glutathione peroxidase 4, pseudogene///glutathione peroxidase 4 | 0.22978626 |
| 1417763_at | Ssr1 | signal sequence receptor, alpha | 0.22852731 |
| 1419041_at | Itfg1 | integrin alpha FG-GAP repeat containing 1 | 0.22592888 |
| 1455788_x_at | Poldip3 | polymerase (DNA-directed), delta interacting protein 3 | 0.22509694 |
| 1448227_at | Grb7 | growth factor receptor bound protein 7 | 0.22311991 |
| 1417417_a_at | Cox6a1 | cytochrome c oxidase subunit VIa polypeptide 1 | 0.21898591 |
| 1423663_at | Flcn | folliculin | 0.19842664 |
| 1452591_a_at | Mzt2 | mitotic spindle organizing protein 2 | 0.19590496 |
| 1416531_at | Gsto1 | glutathione S-transferase omega 1 | 0.19463556 |
| 1456014_s_at | Fermt3 | fermitin family homolog 3 (Drosophila) | 0.19170704 |
| 1429003_at | Snwl | SNW domain containing 1 | 0.18572897 |
| 1433698_a_at | Txnl4a | thioredoxin-like 4A | 0.15615377 |
| 1427865_at | Hbb-b2 | hemoglobin, beta adult minor chain | 0.14273159 |
| 1419094_at | Cyp2c37 | cytochrome P450, family 2. subfamily c, polypeptide 37 | 0.13085943 |
| 1424905_a_at | Slc39a11 | solute carrier family 39 (metal ion transporter), member 11 | 0.10386024 |
| 1439398_x_at | Nsmf | NMDA receptor synaptonuclear signaling and neuronal migration | 0.09924727 |
| | | factor | |
| 1417107_at | Tpd5212 | tumor protein D52-like 2 | 0.09105429 |
| 1425581_s_at | Galnt7 | UDP-N-acetyl-alpha-D-galactosamine:polypeptide N- | 0.07841468 |
| | | acetylgalactosaminyltransferase 7 | |
| 1452743_at | Pole3 | polymerase (DNA directed), epsilon 3 (p17 subunit) | 0.0715764 |
| 1425311_at | 4930432F04Rik | RIKEN cDNA 4930432F04 gene | 0.06937653 |
| 1423875_at | Fam160b1 | family with sequence similarity 160, member B1 | 0.05343816 |

Downregulated Genes

| | | | |
|---|---|---|---|
| 1424337_at | Snx15 | sorting nexin 15 | −0.0552351 |
| 1423540_at | Rbms2 | RNA binding motif, single stranded interacting protein 2 | −0.059713 |
| 1451464_at | Mfap3 | microfibrillar-associated protein 3 | −0.0754051 |
| 1441618_at | Arhgap29 | Rho GTPase activating protein 29 | −0.0792216 |
| 1432426_a_at | Ube2f | ubiquitin-conjugating enzyme E2F (putative) | −0.0793404 |
| 1427292_at | Iglc1///Iglv1/// | immunoglobulin lambda constant 1///immunoglobulin lambda | −0.0835465 |
| | LOC433053 | variable 1///Ig lambda 1 chain c region | |
| 1426479_a_at | Cnpy3 | canopy 3 homolog (zebrafish) | −0.0979648 |
| 1421161_at | Btc | betacellulin, epidermal growth factor family member | −0.1003544 |
| 1425261_at | Cebpg | CCAAT/enhancer binding protein (C/EBP), gamma | −0.1009678 |
| 1453865_a_at | Otud5 | OTU domain containing 5 | −0.1017075 |
| 1452414_s_at | Ccdc86 | coiled-coil domain containing 86 | −0.1093469 |
| 1450437_a_at | Ncam1 | neural cell adhesion molecule 1 | −0.1103669 |
| 1417931_at | Ndst2 | N-deacetylase/N-sulfotransferase (heparan glucosaminyl) 2 | −0.1119908 |
| 1427856_a_at | Igk///Igkv19-93 | immunoglobulin kappa chain complex///immunoglobulin kappa | −0.1138521 |
| | | chain variable 19-93 | |
| 1451838_a_at | Tc2n | tandem C2 domains, nuclear | −0.1157266 |
| 1448341_a_at | Stxbp2 | syntaxin binding protein 2 | −0.1228704 |
| 1450427_at | Chrna6 | cholinergic receptor, nicotinic, alpha polypeptide 6 | −0.1233395 |
| 1436357_at | Ggnbp2os | gametogenetin binding protein 2, opposite strand | −0.1330425 |
| 1420794_at | Art2b | ADP-ribosyltransferase 2b | −0.137948 |
| 1427887_at | Rprd1b | regulation of nuclear pre-mRNA domain containing 1B | −0.1425607 |
| 1416888_at | Fadd | Fas (TNFRSF6)-associated via death domain | −0.1443051 |
| 1424290_at | Osgin2 | oxidative stress induced growth inhibitor family member 2 | −0.1461742 |
| 1438746_at | A530058N18Rik | RIKEN cDNA A530058N18 gene | −0.1501986 |
| 1460728_s_at | Ing4 | inhibitor of growth family, member 4 | −0.1564174 |
| 1427499_at | Zfp81 | zinc finger protein 81 | −0.1576952 |
| 1424107_at | Kifl8a | kinesin family member 18A | −0.1592311 |
| 1426654_at | Zc3hc1 | zinc finger, C3HC type 1 | −0.1621961 |
| 1425919_at | Ndufa12 | NADH dehydrogenase (ubiquinone) 1 alpha subcomplex, 12 | −0.1655454 |
| 1422207_at | Htr5a | 5-hydroxytryptamine (serotonin) receptor 5A | −0.1704528 |
| 1423038_at | Stx6 | syntaxin 6 | −0.1710957 |
| 1429616_at | Zfp91 | zinc finger protein 91 | −0.1728396 |
| 1425454_a_at | Il12a | interleukin 12a | −0.172978 |
| 1419564_at | Zfp467 | zinc finger protein 467 | −0.1737745 |
| 1422964_at | Rad23a | RAD23a homolog (S. cerevisiae) | −0.1772442 |
| 1424484_at | Mob1a | MOB kinase activator 1A | −0.1794421 |
| 1421905_at | Tgs1 | trimethylguanosine synthase homolog (S. cerevisiae) | −0.1820248 |
| 1451629_at | Lbh | limb-bud and heart | −0.1841096 |
| 1426275_a_at | Uxs1 | UDP-glucuronate decarboxylase 1 | −0.186765 |
| 1421578_at | Ccl4 | chemokine (C-C motif) ligand 4 | −0.1891989 |
| 1433816_at | Slc25a51 | solute carrier family 25, member 51 | −0.1902614 |
| 1422171_at | Ptgdr | prostaglandin D receptor | −0.1904976 |
| 1455214_at | Mitf | microphthalmia-associated transcription factor | −0.1909813 |
| 1424532_at | Ylpm1 | YLP motif containing 1 | −0.1932362 |
| 1425660_at | Btbd3 | BTB (POZ) domain containing 3 | −0.1964939 |
| 1431705_a_at | Mcoln2 | mucolipin 2 | −0.1969119 |
| 1436923_at | Rab2b | RAB2B, member RAS oncogene family | −0.1987539 |
| 1419558_at | Mdm4 | transformed mouse 3T3 cell double minute 4 | −0.2023401 |
| 1417412_at | F8a | factor 8-associated gene A | −0.202666 |
| 1434836_at | Nfatc2ip | nuclear factor of activated T cells, cytoplasmic, calcineurin | −0.2061019 |
| | | dependent 2 interacting protein | |
| 1417352_s_at | Snrpa1 | small nuclear ribonucleoprotein polypeptide A′ | −0.2097242 |
| 1427150_at | Kmt2c | lysine (K)-specific methyltransferase 2C | −0.2104669 |
| 1424503_at | Rab22a | RAB22A, member RAS oncogene family | −0.2108491 |
| 1418584_at | Ccnh | cyclin H | −0.2113591 |
| 1438700_at | Fnbp4 | formin binding protein 4 | −0.2149581 |
| 1417898_a_at | Gzma | granzyme A | −0.2149613 |
| 1438721_a_at | Irf3 | interferon regulatory factor 3 | −0.2157688 |
| 1419745_at | Arhgap23 | Rho GTPase activating protein 23 | −0.2160008 |
| 1422919_at | Hrasls | HRAS-like suppressor | −0.2160767 |
| 1424922_a_at | Brd4 | bromodomain containing 4 | −0.22907 |
| 1433860_at | 6030458C11Rik | RIKEN cDNA 6030458C11 gene | −0.2313169 |
| 1421147_at | Terf2 | telomeric repeat binding factor 2 | −0.2324803 |
| 1452497_a_at | Nfatc3 | nuclear factor of activated T cells, cytoplasmic, calcineurin | −0.2335669 |
| | | dependent 3 | |
| 1425434_a_at | Msr1 | macrophage scavenger receptor 1 | −0.2471138 |
| 1438220_at | Foxj3 | forkhead box J3 | −0.2483283 |
| 1425600_a_at | Plcb1 | phospholipase C, beta 1 | −0.2487774 |
| 1438501_at | Rps17 | ribosomal protein S17 | −0.2496664 |
| 1425289_a_at | Cr2 | complement receptor 2 | −0.2506919 |
| 1436388_a_at | 3830406C13Rik | RIKEN cDNA 3830406C13 gene | −0.2537966 |
| 1422175_at | Mmp1a | matrix metallopeptidase 1a (interstitial collagenase) | −0.2543012 |
| 1426907_s_at | Dhx57 | DEAH (Asp-Glu-Ala-Asp/His) box polypeptide 57 | −0.2577509 |
| 1421205_at | Atm | ataxia telangiectasia mutated | −0.2580529 |
| 1425677_a_at | Ank1 | ankyrin 1, erythroid | −0.2639174 |
| 1420433_at | Taf71 | TAF7-like RNA polymerase II, TATA box binding protein (TBP)- | −0.2657971 |
| | | associated factor | |
| 1454086_a_at | Lmo2 | LIM domain only 2 | −0.2678614 |
| 1425802_a_at | Fcrla | Fc receptor-like A | −0.2778881 |
| 1418308_at | Hus1 | Hus1 homolog (S. pombe) | −0.2801646 |
| 1416732_at | Top2b | topoisomerase (DNA) II beta | −0.2816183 |
| 1427113_s_at | Ttl | tubulin tyrosine ligase | −0.2820298 |
| 1427031_s_at | Spice 1 | spindle and centriole associated protein 1 | −0.2823499 |
| 1420369_a_at | Csn2 | casein beta | −0.2830043 |
| 1447977_x_at | 0610010B08Rik///Gm11007/// | RIKEN cDNA 0610010B08 gene///predicted gene 11007/// | −0.2834026 |
| | Gm14308///Gm14430/// | predicted gene 14308///predicted gene 14430///predicted gene | |
| | Gm14434///Gm4724 | 14434///predicted gene 4724 | |
| 1453473_a_at | Dynlt1-ps1///Dynlt1a/// | dynein light chain Tctex-type 1, pseuodogene 1///dynein light | −0.2840156 |
| | Dynlt1b///Dynlt1c///Dynlt1f | chain Tctex-type 1A///dynein light chain Tctex-type 1B///dynein | |
| | | light chain Tctex-type 1C///dynein light chain Tctex-type 1F | |
| 1451684_a_at | Bicd1 | bicaudal D homolog 1 (Drosophila) | −0.2849648 |
| 1438502_x_at | Rps17 | ribosomal protein S17 | −0.2862873 |
| 1417329_at | Slc23a2 | solute carrier family 23 (nucleobase transporters), member 2 | −0.2872516 |
| 1454607_s_at | Psat1 | phosphoserine aminotransferase 1 | −0.2905989 |
| 1421183_at | Tex12 | testis expressed gene 12 | −0.2957092 |
| 1456117_at | Rrp1b | ribosomal RNA processing 1 homolog B (S. cerevisiae) | −0.2982989 |
| 1450091_at | Ighmbp2 | immunoglobulin mu binding protein 2 | −0.3035937 |
| 1418334_at | Dbf4 | DBF4 homolog (S. cerevisiae) | −0.3156701 |
| 1423103_at | Rfx5 | regulatory factor X, 5 (influences HLA class II expression) | −0.318144 |
| 1452839_at | Dph5 | DPH5 homolog (S. cerevisiae) | −0.3182936 |
| 1426544_a_at | Ttc14 | tetratricopeptide repeat domain 14 | −0.3188792 |
| 1451349_at | Efcab7 | EF-hand calcium binding domain 7 | −0.3233028 |
| 1426536_at | Ice2 | interactor of little elongation complex ELL subunit 2 | −0.3237006 |
| 142493l_s_at | Iglc1///Iglv1 | immunoglobulin lambda constant 1///immunoglobulin | −0.3238676 |
| | | lambda variable 1 | |
| 1433758_at | Nisch | nischarin | −0.3254956 |
| 1418755_at | Tbx15 | T-box 15 | −0.3285867 |
| 1459885_s_at | Cox7c///LOC102642884 | cytochrome c oxidase subunit VIIc///cytochrome | −0.3308862 |
| | | c oxidase subunit 7C, mitochondrial | |
| 1425585_at | Med12 | mediator complex subunit 12 | −0.3318173 |
| 1431830_at | Zfp329 | zinc finger protein 329 | −0.3342973 |
| 1418830_at | Cd79a | CD79A antigen (immunoglobulin-associated alpha) | −0.3399401 |
| 1420509_at | LOC102641936///Srfbp1 | serum response factor-binding protein 1///serum | −0.3404931 |
| | | response factor binding protein 1 | |
| 1421033_a_at | Tcerg1 | transcription elongation regulator 1 (CA150) | −0.343087 |
| 1427614_at | Pip | prolactin induced protein | −0.3445432 |
| 1436222_at | Gas5 | growth arrest specific 5 | −0.3451841 |
| 1427553_at | Gm16489 | predicted gene 16489 | −0.3480588 |
| 1424936_a_at | Dnah8 | dynein, axonemal, heavy chain 8 | −0.3586105 |
| 1460242_at | Cd55 | CD55 molecule, decay accelerating factor for complement | −0.3602501 |
| 1424504_at | Rab22a | RAB22A, member RAS oncogene family | −0.3639248 |
| 1422961_at | Nat3 | N-acetyltransferase 3 | −0.3787963 |
| 1455904_at | Gas5///Snord47 | growth arrest specific 5///small nucleolar RNA, | −0.3802181 |
| | | C/D box 47 | |
| 1435064_a_at | Tmem27 | transmembrane protein 27 | −0.3877336 |
| 1418608_at | Calml3 | calmodulin-like 3 | −0.4082519 |
| 1460407_at | Spib | Spi-B transcription factor (Spi-1/PU.1 related) | −0.4214451 |
| 1427575_at | Fbxw14 | F-box and WD-40 domain protein 14 | −0.4227238 |
| 1416055_at | Amy2a2///Amy2a3/// | amylase 2a2///amylase 2a3///amylase 2a4///amylase 2a5 | −0.4234871 |
| | Amy2a4///Amy2a5 | | |
| 1422940_x_at | Serpinb3b///Serpinb3c | serine (or cysteine) peptidase inhibitor, clade | −0.4263653 |
| | | B (ovalbumin), member 3B///serine (or cysteine) | |
| | | peptidase inhibitor, clade B, member 3C | |
| 1441992_at | Rab14 | RAB14, member RAS oncogene family | −0.4295766 |
| 1424852_at | Mef2c | myocyte enhancer factor 2C | −0.4301503 |
| 1450595_at | Vmn1r10///Vmn1r9 | vomeronasal 1 receptor 10///vomeronasal 1 receptor 9 | −0.4630468 |
| 1433946_at | Zik1 | zinc finger protein interacting with K protein 1 | −0.4722573 |
| 1453864_at | Rdh14 | retinol dehydrogenase 14 (all-trans and 9-cis) | −0.472489 |
| 1452997_at | Gm21811 | predicted gene, 21811 | −0.4845378 |
| 1448821_at | Tyr | tyrosinase | −0.5015319 |
| 1427256_at | Vcan | versican | −0.5905388 |
| 1435331_at | Pyhin1 | pyrin and HIN domain family, member 1 | −0.5912079 |
| 1424537_at | Gm10921///Gm10922///Gm14345/// | predicted gene 10921///predicted gene 10922/// | −0.595637 |
| | Gm14346///Gm14347///Gm14351/// | predicted gene 14345///predicted gene 14346/// | |
| | Gm14367///Gm14374///Gm21608/// | predicted gene 14347///predicted gene 14351/// | |
| | Gm21645///Gm21681///Gm21699/// | predicted gene 14367///predicted gene 14374/// | |
| | Gm21950///Gm21951///Gm2777/// | predicted gene, 21608///predicted gene, | |
| | Gm2784///Gm2799///Gm2825/// | 21645///predicted gene, 21681///predicted gene, | |
| | Gm2863///Gm2913///Gm2927/// | 21699///predicted gene, 21950///predicted gene, | |
| | Gm2964///Gm3701///Gm3706/// | 21951///predicted gene 2777///predicted gene | |
| | Gm3750///Gm3763///Gm5925/// | 2784///predicted gene 2799///predicted gene | |
| | Gmcl11///LOC100048813 | 2825///predicted gene 2863///predicted gene | |
| | | 2913///predicted gene 2927///predicted gene | |
| | | 2964///predicted gene 3701///predicted gene | |
| | | 3706///predicted gene 3750///predicted gene | |
| | | 3763///germ cell-less homolog 1 family | |
| | | pseudogene///germ cell-less homolog 1 | |
| | | (Drosophila)-like///germ cell-less-like | |
| 1423226_at | Ms4a1 | membrane-spanning 4-domains, subfamily A, member 1 | −0.6014881 |
| 1427346_at | Gm10439///Gm15080///Gm15085/// | predicted gene 10439///predicted gene 15080/// | −0.6588143 |
| | Gm15093///Gm15107///Gm15109/// | predicted gene 15085///predicted gene 15093/// | |
| | Gm15114///Gm15128///Luzp4///Ott | predicted gene 15107///predicted gene 15109/// | |
| | | predicted gene 15114///predicted gene 15128/// | |
| | | leucine zipper protein 4///ovary testis transcribed | |

The 128 genes from Classifier 1 that were found to be confounded by the presence of immunosuppression clustered into immune-related GO biological pathways related to response to lipopolysaccharide, positive regulation of apoptotic process, response to bacterium, positive regulation of B cell proliferation, positive regulation of nitric oxide biosynthetic process, interferon-gamma production, and regulation of the Wnt signaling pathway. The 169 genes ultimately included only in the final classifier (Classifier 2), and thus stable across both immunocompetent and different immunocompromised states, were found to be enriched for several biological pathways: immune system process, B cell activation, B cell proliferation, regulation of the inflammatory response, and positive regulation of type I interferon-mediated signaling pathway.

### Example 6. Validation of Fungal Response Genes in a Murine Model of Invasive Candidiasis

In order to further assess the role of these genes in host antifungal responses, further analysis was performed to investigate whether conserved genes characteristic of the host response to IA were also able to identify cases of another fungal infection. Transcriptomic data from peripheral blood samples were analyzed in a murine model of invasive candidiasis (Zaas et al. (2010), “Blood gene expression signatures predict invasive candidiasis,” Science translational medicine, 2(21):21ra17). When compared to uninfected controls, the transcriptomic responses to Aspergillus (a total of 2,826 probe sets identified for Aspergillus vs. Control) and Candida (a total of 7,925 probe sets identified Candida vs. Control) shared 1,215 significantly differentially expressed genes (illustration of overlap not shown here; see Supplementary Fig. S2 in Steinbrink et al., 2020). Additionally, IPA of the intersection of significant probes from the differential expression analysis demonstrated top biologic functions related to immunity including quantity/cell movement/binding of leukocytes, quantity/cell movement of lymphocytes, proliferation of immune cells, and immune response of cells. These shared fungal response genes reflect immune related pathways that typify the response to IA see in our initial model here. Twenty-four percent of the genes in the Aspergillus classifier (Classifier 1) also separated mice infected with Candida from healthy controls with a high degree of accuracy (see Supplementary Fig. S3 and S4 in Steinbrink et al., 2020) data not shown here; see, suggesting that many of the IA-associated genes described herein are common to the murine response to fungal infections.

### Example 7. Generation and Evaluation of a Host Transcriptomic Signature of Invasive Aspergillosis from Samples of at-Risk Immunocompromised Subjects

A repository of clinical specimens from almost 900 enrollees with IA as well as those at risk for infection, developed by the NIAID-funded Aspergillus Technology Consortium (AsTeC), will be used for the analysis of new fungal diagnostic methods. These specimens were collected from the University of Florida, Duke University, and Brigham and Women's/Dana Farber Cancer Institute for three high-risk patient populations—bone marrow transplant recipients, hematologic malignancy patients, and lung transplant recipients.

The banked peripheral blood samples will be used through the existing relationship with AsTeC from a subset of 40 of these subjects with proven and probable IA. RNA sequencing will be performed on these subjects for samples obtained both at the time of infection diagnosis as well as a pre-infection baseline. Since the eventual goal is to utilize the IA signature in subjects with undifferentiated febrile illness, previously banked samples from subjects with bacterial and viral infection will also be included as clinical comparators. Bayesian approaches will be used with the curated dataset to define patterns of gene expression or ‘signatures’ that separate IA from other clinical syndromes with similar presentations. The diagnostic performance of these signatures will then be compared with existing standard of care serum fungal markers (i.e., galactomannan).

The ultimate aim of this analysis will be to develop improved companion fungal diagnostics, particularly for the immunocompromised population. These data will not only provide proof of principle that a new diagnostic test for IA can be developed, but will also provide pilot data for a later trial that can translate this type of test to the clinical arena.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of treating an Aspergillus infection in a subject, the method comprising:
(a) measuring, the gene expression levels of two or more aspergillosis versus reference (“AvR”) genes selected from the group consisting of gene markers listed in Table 2 in a biological sample obtained from the subject; and
(b) administering an effective amount of an antifungal treatment to the subject identified as having an Aspergillus infection based on comparison of the gene expression levels of the two or more AvR genes with reference gene expression levels of the AvR genes in a reference sample having a known Aspergillus infection classification.

2. A method of treating an Aspergillus infection in a subject, the method comprising:
(a) selecting a subject who has been classified as having an Aspergillus infection based on the gene expression levels of two or more aspergillosis versus reference (“AvR”) genes selected from the group consisting of gene markers listed in Table 2 relative to reference expression levels determined for the AvR genes in a reference sample having a known Aspergillus infection classification; and
(b) administering to the subject an effective amount of an antifungal treatment.

3. A method of determining the presence of an Aspergillus infection in a subject, the method comprising:
(a) measuring, the gene expression levels of two or more aspergillosis versus reference (“AvR”) genes selected from the group consisting of gene markers listed in Table 2 in a biological sample obtained from the subject; and
(b) identifying the subject as having an Aspergillus infection based on comparison of the gene expression levels of the two or more AvR genes in the biological sample to reference expression levels determined for the AvR genes in a reference sample having a known Aspergillus infection classification.

4. The method of claim 3, wherein the gene expression levels of all the gene markers listed in Table 2 are measured.

5. The method of claim 3, wherein the biological sample is blood, serum, plasma, lung tissue, or a sample that is obtained using a nasal swab, a nasopharyngeal swab, an oropharyngeal swab, a buccal swab, a broncho-alveolar lavage, or a tracheobronchial aspirate.

6. The method of claim 3, wherein measuring the gene expression level(s) comprises performing polymerase chain reaction (PCR), isothermal amplification, next generation sequencing (NGS), mass spectrometry, microarray analysis, enzyme-linked immunosorbent assay (ELISA), Northern blot, or serial analysis of gene expression (SAGE).

7. The method of claim 3, wherein the measured gene expression levels are RNA expression levels measured by polymerase chain reaction (PCR) or microarray analysis, or a combination thereof.

8. The method of claim 3, wherein the reference sample having a known Aspergillus infection classification is from a subject that does not have an Aspergillus infection.

9. The method of claim 3, wherein the reference sample having a known Aspergillus infection classification is from a subject that has an Aspergillus infection.

10. The method of claim 3, wherein the subject is suspected of having a fungal infection.

11. The method of claim 3, wherein the subject is suspected of having an Aspergillus infection.

12. The method of claim 3, wherein the subject has acute respiratory illness symptoms.

13. The method of claim 3, wherein the subject has symptoms of a fungal infection.

14. The method of claim 3, wherein the subject has symptoms of an Aspergillus infection.

15. The method of claim 3, wherein the subject is immunocompromised.

16. The method of claim 3, wherein the subject has received an organ transplantation or stem cell transplantation.

17. The method of claim 3, wherein the subject has been previously treated or is being treated with steroids.

18. The method of claim 3, wherein the subject has been previously treated or is being treated with chemotherapy.

19. The method of claim 3, wherein the subject previously has had a viral acute respiratory infection.
